(19) United States
(12) Patent Application Publication
(10) Pub. No.: US 2007/0081983 A1
(43) Pub. Date: Apr. 12, 2007

(54) Isolation of smooth muscle cells and tissue-engineered vasculature containing the isolated cells

(75) Inventors: Stelios T. Andreadis, Williamsville, NY (US); Jin Yu Liu, Amherst, NY (US)

Correspondence Address: NIXON PEABODY LLP - PATENT GROUP, CLINTON SQUARE, P.O. BOX 31051, ROCHESTER, NY, 14603-1051, US

(73) Assignee: The Research Foundation of State University of New York, Amherst, NY (US)

(21) Appl. No.: 11/523,474

(22) Filed: Sep. 19, 2006

Related U.S. Application Data
(60) Provisional application No. 60/718,813, filed on Sep. 20, 2005.

(51) Int. Cl. A61K 48/00; C12Q 1/00; C12N 5/08

(52) U.S. Cl. 424/93.21; 435/4; 435/455; 435/366

(57) ABSTRACT

The present invention is directed to a method of isolating smooth muscle cells or progenitors thereof from a mixed population of cells. A preparation of isolated smooth muscle cells or progenitors thereof, where the smooth muscle cells or progenitors thereof constitute at least 90% of the preparation, is also disclosed. The present invention is also directed to a method of producing a tissue-engineered vascular vessel containing the preparation of isolated smooth muscle cells or progenitors thereof. The resulting tissue-engineered vascular vessel and a method of producing a tissue-engineered vascular vessel for a particular patient are also disclosed.

This application claims the priority benefit of U.S. Provisional Patent Application Ser. No. 60/718,813, filed Sep. 20, 2005, which is hereby incorporated by reference in its entirety.

### FIELD OF THE INVENTION

The present invention relates to isolation of functional smooth muscle cells using tissue specific promoters and to tissue-engineered vasculature containing the isolated smooth muscle cells.

### BACKGROUND OF THE INVENTION

Cardiovascular disease is the leading cause of mortality in western countries and around the world, increasing the demand for small diameter blood vessels as replacement grafts. Although venous grafts are currently the golden standard, they suffer several major disadvantages: (i) availability may be limited, especially for repeat grafting procedures; (ii) there is pain and discomfort associated with the donor site; (iii) the replicative capacity of cells from older donors is limited (Poh et al., “Blood Vessels Engineered from Human Cells,” Lancet 365(9477):2122-2124 (2005); McKee et al., “Human Arteries Engineered In Vitro,” EMBO Rep. 4(6):633-638 (2003)); and (iv) the ten-year failure rate is high (Gaudino et al., “Arterial Versus Venous Bypass Grafts in Patients with In-Stent Restenosis,” Circulation 112(9 Suppl): 1265-269 (2005)). Tissue engineering can provide an alternative to existing technologies by providing autologous tissue engineered vessels (“TEV”) for vascular repair and regeneration.

Three major approaches have been proposed for tissue engineering of vascular grafts: (i) decellularized blood vessels; (ii) cell sheet engineering; and (iii) biodegradable scaffolds from natural or synthetic polymers. Scaffolds derived from decellularized blood vessels have been implanted directly or after addition of endothelial and smooth muscle cells to improve patency and vascular reactivity (Huynh et al., “Remodeling of an Acellular Collagen Graft Into a Physiologically Responsive Neovessel,” Nature Biotechnology 17(11):1083-1086 (1999); Bader et al., “Engineering of Human Vascular Aortic Tissue Based on a Xenogeneic Starter Matrix,” Transplantation 70(1):7-14 (2000); Kaushal et al., “Functional Small-Diameter Neovessels Created Using Endothelial Progenitor Cells Expanded Ex Vivo,” Nature Medicine 7(9):1035-1040 (2001)). Cell sheet engineering does not employ a scaffold, but relies on the ability of the cells to form highly interconnected sheets when grown to high densities. When these sheets were wrapped around a mandrel and cultured for several weeks, they yielded multi-layered cylindrical tissues with high mechanical strength and vascular reactivity (L'Heureux et al., “A Completely Biological Tissue-Engineered Human Blood Vessel,” FASEB Journal 12(1):47-56 (1998); L'Heureux et al., “A Human Tissue-Engineered Vascular Media: A New Model for Pharmacological Studies of Contractile Responses,” FASEB Journal 15(2):515-524 (2001)). Finally, synthetic and natural polymers have been used as scaffolds to support cell growth and provide mechanical support necessary for implantation. Polyglycolic acid (“PGA”) and co-polymers of PGA with poly-L-lactic acid, polycaprolactone, or poly-4-hydroxybutyrate have been used with various degrees of success (Niklason et al., “Functional Arteries Grown In Vitro,” Science 284:489-493 (1999); Niklason et al., “Morphologic and Mechanical Characteristics of Engineered Bovine Arteries,” J. Vase. Surg. 33(3):628-638 (2001); Kim et al., “Engineered Smooth Muscle Tissues: Regulating Cell Phenotype with the Scaffold,” Exp. Cell. Res. 251 (2):318-328 (1999); Shin'oka et al., “Transplantation of a Tissue-Engineered Pulmonary Artery,” New England Journal of Medicine 344(7):532-533 (2001); Watanabe et al., “Tissue-Engineered Vascular Autograft: Inferior Versa Cava Replacement in a Dog Model,” Tissue Engineering 7(4):429-439 (2001); Lee et al., “Elastic Biodegradable Poly(Glycolide-Co-Caprolactone) Scaffold for Tissue Engineering,” J. Biomed. Mater. Res. A 66(1):29-37 (2003); Hoerstrup et al., “Living, Autologous Pulmonary Artery Conduits Tissue Engineered from Human Umbilical Cord Cells,” Ann. Thorac. Surg. 74(1):46-52 (2002); Wake et al., “Fabrication of Pliable Biodegradable Polymer Foams to Engineer Soft Tissues,” Cell Transplant 5(4):465-473 (1996)). Natural biomaterials such as collagen and fibrin have also been employed, because they can polymerize in the presence of cells and contain inherent biological signals that influence cellular activity (Barocas et al., “Engineered Alignment in Media Equivalents: Magnetic Prealignment and Mandrel Compaction,” J. Biomech. Eng. 120(5):660-666 (1998); Seliktar et al., “Mechanical Strain-Stimulated Remodeling of Tissue-Engineered Blood Vessel Constructs,” Tissue Eng. 9(4):657-666 (2003); Stegemann et al., “Altered Response of Vascular Smooth Muscle Cells to Exogenous Biochemical Stimulation in Two- and Three-Dimensional Culture,” Experimental Cell Research 283(2):146-155 (2003)). It has recently been demonstrated that fibrin-based small-diameter TEV can be implanted in an ovine animal model using fibrin hydrogels (Swartz et al., “Engineering of Fibrin-Based Functional and Implantable Small-Diameter Blood Vessels,” Am. J. Physiol. Heart Circ. Physiol. 288(3):H1451-1460 (2005)). After only two weeks in culture, TEV exhibited significant reactivity in response to several vasodilators and vasoconstrictors and developed considerable mechanical strength to withstand interpositional implantation in the jugular veins of lambs, where they remained patent for 15 weeks and displayed significant matrix remodeling (Swartz et al., “Engineering of Fibrin-Based Functional and Implantable Small-Diameter Blood Vessels,” Am. J. Physiol. Heart Circ. Physiol. 288(3):H1451-1460 (2005)).

Despite significant progress toward development of biomaterials and methods to cultivate 3D vascular constructs, cell sourcing remains a major problem, since isolation of smooth muscle and endothelial cells from autologous vessels injures the donor site and may also be limited by the health of the patient. In addition, adult somatic cells were shown to exhibit limited replicative capacity, especially when they originated from older donors who are the ones more likely to suffer from cardiovascular disease (Poh et al., “Blood Vessels Engineered from Human Cells,” Lancet, 365(9477):2122-2124 (2005); McKee et al., “Human Arteries Engineered In Vitro,” EMBO Rep. 4(6):633-638 (2003)). Therefore, an autologous source of progenitor vascular cells with high proliferative capacity is necessary to enable isolation and expansion of cells to large numbers necessary for preparation of TEV.

Stem cells have tremendous potential as an autologous, non-immunogenic cell source for tissue regeneration. Specifically, adult stem cells provide a promising alternative and can be isolated from the same patient, which avoids immune rejection and long-term immunosuppression. For example, bone marrow-derived stem cells have high proliferation potential, can home into sites of vascular injury where they differentiate into vascular cells (Galmiche et al., “Stromal Cells from Human Long-Term Marrow Cultures are Mesenchymal Cells that Differentiate Following a Vascular Smooth Muscle Differentiation Pathway,” Blood 82(1):66-76 (1993); Shimizu et al., “Host Bone-Marrow Cells Are a Source of Donor Intimal Smooth- Muscle-Like Cells in Murine Aortic Transplant Arteriopathy,” Nat. Med. 7(6):738-741 (2001); Hillebrands et al., “Origin of Neointimal Endothelium and Alpha-Actin-Positive Smooth Muscle Cells in Transplant Arteriosclerosis,” J. Clin. Invest. 107(11):1411-1422 (2001); Han et al., “Circulating Bone Marrow Cells Can Contribute to Neointimal Formation,” J. Vase. Res. 38(2):113-119 (2001); Sata et al., “Hematopoietic Stem Cells Differentiate Into Vascular Cells that Participate in the Pathogenesis of Atherosclerosis,” Nat. Med. 8(4):403-409 (2002)), and can even be allografted to histocompatible receivers (Liechty et al., “Human Mesenchymal Stem Cells Engraft and Demonstrate Site-Specific Differentiation After In Utero Transplantation In Sheep,” Nat. Med. 6(11):1282-1286 (2000)). Finally, adult stem cells are not compounded by the ethical considerations of embryonic stem cells and they are readily available for research.

Several animal studies have suggested that bone marrow progenitor cells can infiltrate the atherosclerotic intima and differentiate to form smooth muscle and endothelial cells within the atherosclerotic plaque (Hillebrands et al., “Origin of Neointimal Endothelium and Alpha-Actin-Positive Smooth Muscle Cells in Transplant Arteriosclerosis,” J. Clin. Invest. 107(11):1411-1422 (2001); Han et al., “Circulating Bone Marrow Cells Can Contribute to Neointimal Formation,” J. Vase. Res. 38(2):113-119 (2001); Sata et al., “Hematopoietic Stem Cells Differentiate Into Vascular Cells that Participate In the Pathogenesis of Atherosclerosis,” Nat. Med. 8(4):403-409 (2002); Saiura et al., “Circulating Smooth Muscle Progenitor Cells Contribute to Atherosclerosis,” Nat. Med. 7(4):382-383 (2001)). Smooth muscle cells from sex-mismatched (Caplice et al., “Smooth Muscle Cells in Human Coronary Atherosclerosis Can Originate From Cells Administered at Marrow Transplantation,” Proc. Natl. Acad. Sci. USA 100(8):4754-4759 (2003)) or β-galactosidase-expressing (Shimizu et al., “Host Bone-Marrow Cells Are a Source of Donor Intimal Smooth-Muscle-Like Cells in Murine Aortic Transplant Arteriopathy,” Nat. Med. 7(6):738-741 (2001)) bone marrow transplants were recruited to a much larger extent to diseased as compared to healthy blood vessels. These studies suggest that there are smooth muscle progenitor cells in the bone marrow and peripheral blood.

Several investigators have attempted to culture smooth muscle cells from bone marrow mononuclear cells by stimulation with cytokines and growth factors such as PDGF-BB or TGF-β1 (Simper et al., “Smooth Muscle Progenitor Cells In Human Blood,” Circulation 106(10):1199-1204 (2002); Le Ricousse-Roussanne et al., “Ex Vivo Differentiated Endothelial and Smooth Muscle Cells from Human Cord Blood Progenitors Home to the Angiogenic Tumor Vasculature,” Cardiovasc. Res. 62(1):176-184 (2004); Cho et al., “Small-Diameter Blood Vessels Engineered With Bone Marrow-Derived Cells,” Ann. Surg. 241(3):506-515 (2005)). Although soluble factors in the medium can direct differentiation of a fraction of cells toward the smooth muscle cell (“SMC”) lineage, these approaches have not demonstrated isolation of a pure population of functional, contractile, SMC. One study used an SM22 promoter to select for SMC from bone marrow mononuclear cells (Kashiwakura et al., “Isolation of Bone Marrow Stromal Cell-Derived Smooth Muscle Cells by a Human SM22alpha Promoter: In Vitro Differentiation of Putative Smooth Muscle Progenitor Cells of Bone Marrow,” Circulation 107(16):2078-2081 (2003)). Interestingly, cells with an active SM22 promoter expressed neither immature nor mature SMC markers. Only after G418 selection for 25 days were clones of cells that expressed SMC markers identified, suggesting that merely a fraction of cells with active SM22 promoter expressed SMC markers. In addition, functional properties of these cells, such as gel compaction or vascular reactivity, were not investigated, and, therefore, it was not clear whether these cells could be used for vascular tissue engineering.

The present invention is directed to overcoming the limitations in the prior art.

### SUMMARY OF THE INVENTION

One aspect of the present invention is directed to a method of isolating smooth muscle cells or progenitors thereof from a mixed population of cells. This method involves selecting an enhancer/promoter which functions in the smooth muscle cells or progenitors thereof. A nucleic acid molecule encoding a marker protein under control of the enhancer/promoter is introduced into the mixed population of cells. The smooth muscle cells or progenitors thereof are allowed to express the marker protein. The smooth muscle cells or progenitors thereof are separated from the mixed population of cells based on expression of the marker protein.

Another aspect of the present invention is directed to a preparation of isolated smooth muscle cells or progenitors thereof, where the smooth muscle cells or progenitors thereof constitute at least 90% of said preparation.

A further aspect of the present invention is directed to a method of producing a tissue-engineered vascular vessel. This method involves providing a vessel-forming fibrin mixture containing fibrinogen, thrombin, and the above-described preparation of isolated smooth muscle cells or progenitors thereof. The vessel-forming fibrin mixture is molded into a fibrin gel having a tubular shape. The fibrin gel having a tubular shape is incubated in a medium suitable for growth of the cells under conditions effective to produce a tissue-engineered vascular vessel.

Yet another aspect of the present invention is directed to a tissue-engineered vascular vessel containing a gelled fibrin mixture having fibrinogen, thrombin, and the preparation of isolated smooth muscle cells or progenitors thereof as described above. The gelled fibrin mixture has a tubular shape.

Yet a further aspect of the present invention is directed to a method of producing a tissue-engineered vascular vessel for a particular patient. This method involves providing a vessel-forming fibrin mixture containing fibrinogen, thrombin, and the preparation of isolated smooth muscle cells or progenitors described above, at least one of which is autologous to the patient. The vessel-forming fibrin mixture is molded into a fibrin gel having a tubular shape. The fibrin gel having a tubular shape is incubated in a medium suitable for growth of the cells under conditions effective to produce a tissue-engineered vascular vessel for a particular patient. The tissue-engineered vascular vessel is implanted into the particular patient.

The present invention is directed to a highly purified population of bone-marrow derived smooth muscle cells (“BM-SMC”) obtained using fluorescence-activated cell sorting (“FACS”) to separate bone marrow mononuclear cells (“BM-MNC”) that express enhanced green fluorescent protein (“EGFP”) under the control of the smooth muscle α-actin (“SMαA”) promoter. These cells exhibit high proliferation potential and express early, intermediate, and late markers of vascular smooth muscle cells. BM-SMC are embedded in fibrin hydrogels, which are polymerized around 4 mm diameter mandrels to engineer cylindrical TEV (“BM-TEV”). These engineered blood vessels exhibit vascular reactivity in response to KCl and norepinephrine (“NE”) and mechanical properties that are comparable to those of TEV from vascular smooth muscle cells. Endothelial cells are also isolated from BM-MNC and are seeded in the lumen of BM-TEV that are subsequently implanted into a subject. At 5-8 weeks post-implantation, explanted BM-TEV display a confluent endothelial monolayer, circumferential alignment of smooth cells in close proximity to the lumen, and remarkable matrix remodeling. Specifically, BM-TEV show high levels of collagen and fibrillar elastin very similar to native veins. Accordingly, progenitor cells can be used to engineer vasoreactive and implantable TEV, thus providing an unlimited supply of highly proliferative, autologous cells for cardiovascular tissue engineering.

### BRIEF DESCRIPTION OF THE DRAWINGS

FIGS. 1A-E show isolation of BM-SMC from bone marrow using a tissue specific promoter according to one embodiment of the present invention. FIG. 1A illustrates a smooth muscle alpha actin promoter (“SMα-EGFP”), which was amplified from rat genomic DNA using PCR and ligated onto promoterless vector pEGFPI between the XhoI and BamHI sites. FIG. 1B shows the process for transfecting mononuclear cells (“MNC”) with the SMα-EGFP plasmid and the observation of fluorescent cells two days later using a fluorescent microscope. FIGS. 1C-E are graphs showing separation of EGFP+cells by fluorescence activated cell sorting.

FIGS. 2A-H show that isolated BM-SMC of the present invention display morphological and biochemical characteristic of vascular smooth muscle cells (“V-SMC”). FIG. 2A is a photograph showing BM-SMC, which are spindle-shaped with a well-organized actin network. FIGS. 2B-C are photographs showing the results of immunostaining demonstrating that BM-SMC expressed smooth muscle α-actin (FIG. 2B) and calponin (FIG. 2C). FIG. 2D shows a Western blot for α-actin and calponin. Lane 1: BM-SMC; lane 2: human keratinocytes; lane 3: V-SMC; lane 4: V-EC. Beta-actin served as loading control (n=2). FIGS. 2E-H are flow cytometry graphs showing that BM-SMC expressed integrins α5 and β1 to a similar extent as V-SMC.

FIGS. 3A-E show that bone marrow-derived endothelial cells (“BM-EC”) displayed morphological and biochemical characteristics of vascular endothelial cells (“V-EC”). BM-EC were isolated from bone marrow and cultured in endothelial growth medium (“EGM”), supplemented with epidermal growth factor (“EGF”), basic fibroblast growth factor (“bFGF”), and fibronectin. FIG. 3A is a photograph showing that BM-EC displayed cobblestone morphology and formed well-organized confluent monolayers (magnification 10×). FIG. 3B is a photograph showing that BM-EC stained positive for Dil-Ac-LDL. Immunocytochemistry showed strong staining of BM-EC for CD31 (FIG. 3C); CD 144 (FIG. 3D); and vWF (magnification 40×) (FIG. 3E).

FIGS. 4A-E show that BM-TEV from BM-SMC, according to the present invention, displayed similar morphologic and biochemical characteristics as TEVs from V-SMC. FIG. 4A illustrates how BM-SMC were embedded in fibrin hydrogels and cultured around a 4-mm mandrel for 2 weeks to form cylindrical tubes with 0.5 mm wall thickness. FIGS. 4B-C are photographs demonstrating that by hematoxylin and eosin (“H&E”) staining, TEV from BM-SMC (FIG. 4B) are distributed uniformly compared to TEV from V-SMC (FIG. 4C) (magnification 10×). FIGS. 4D-E are photographs of immunostaining of BM-TEV and TEV from V-SMC for smooth muscle α-actin (FIG. 4D) and calponin (FIG. 4E) (magnification 40×).

FIGS. 5A-E are graphs showing that BM-TEVs developed considerable mechanical strength and vascular reactivity. TEV from BM-SMC or V-SMC were cultured around 4-mm mandrels for 2 weeks. Mechanical strength and vascular reactivity were measured using an isolated tissue bath system. FIG. 5A is a graph showing force-length curve; FIG. 5B is a graph showing break force; FIG. 5C is a graph showing toughness; FIG. 5D is a graph showing elastic modulus; and FIG. 5E is a graph showing reactivity in response to KCl (118 mM.) or NE (10⁻⁶ M). Data are presented as mean±standard deviation of samples in three independent experiments, each with triplicate samples. The symbol (*) indicates p<0.05 between samples as indicated and (#) indicates a very small value close to zero.

FIGS. 6A-H show that explanted TEVs exhibited similar morphology and deposition of ECM as compared to a native jugular vein. TEVs from BM-SMC were implanted in the jugular vein of 8-week old lambs. In the photographs of FIGS. 6A-B, H&E of native vein and explanted BM-TEV show that BM-TEV contained multiple layers of smooth muscle cells that were overlaid by continuous monolayer of endothelial cells. In the photographs of FIGS. 6C-D, immunohistochemistry showed vWF and BM-SMC for smooth muscle α-actin. In the photographs of FIGS. 6E-F, Mason's trichrome showed abundant collagen in both the native jugular vein (FIG. 6E) and explanted BM-TEV (FIG. 6F). In the photographs of FIGS. 6G-H, Verhoff's elastin stain showed abundant expression and fibrillar organization of elastin (black lines) in both native tissue (FIG. 6G) and explanted BM-TEV (FIG. 6H). Luminal surface is at the top of each panel and blood flows in the direction that crosses the plane of the page (magnification 40×).

FIGS. 7A-B are photographs of immunocytochemistry experiments showing that smooth muscle precursor cells isolated from hair follicle express the vascular smooth muscle cell specific markers α-actin (FIG. 7A) and calponin (FIG. 7B).

FIGS. 8A-D are graphs showing mechanical properties of blood vessels tissue-engineered using hair follicle-derived smooth muscle cells (“HF-SMC”) as a cell source and fibrin hydro gel as a scaffold, compared to TEVs from vascular smooth muscle cells.

FIG. 9 is a graph showing contractility (vasoreactivity to KCl) of HF-TEV compared to V-TEVs.

### DETAILED DESCRIPTION OF THE INVENTION

One aspect of the present invention is directed to a method of isolating smooth muscle cells or progenitors thereof from a mixed population of cells. This method involves selecting an enhancer/promoter which functions in the smooth muscle cells or progenitors thereof. A nucleic acid molecule encoding a marker protein under control of the enhancer/promoter is introduced into the mixed population of cells. The smooth muscle cells or progenitors thereof are allowed to express the marker protein. The smooth muscle cells or progenitors thereof are separated from the mixed population of cells based on expression of the marker protein.

The cells of particular interest according to the present invention are smooth muscle cells or progenitor cells thereof. Any of these cells which one desires to separate from a mixed population of cells can be selected in accordance with the present invention, as long as a promoter specific for the chosen cell is available. “Specific,” as used herein to describe a promoter, means that the promoter functions only in the chosen cell type. A chosen cell type can refer to smooth muscle cells or different stages in the developmental cycle of a progenitor of a smooth muscle cell. For example, the chosen cell may be committed to a particular adult cell phenotype and the chosen promoter only functions in that progenitor cell (i.e. the promoter does not function in adult cells). Although committed and uncommitted progenitor cells may both be considered progenitor cells, these cells are at different stages of progenitor cell development and can be separated according to the present invention if the chosen promoter is specific to the particular stage of the progenitor cell. Those of ordinary skill in the art can readily determine a cell of interest to select based on the availability of a promoter specific for that cell of interest.

Promoters suitable for carrying out this aspect of the present invention include, without limitation, smooth muscle α-actin promoter, SM22 promoter, caldesmon promoter, myosin heavy chain promoter, calponin promoter, and smoothelin promoter.

Having determined the cell of interest and selected a promoter specific for the cell of interest, a nucleic acid molecule encoding a marker protein, preferably a green fluorescent protein (“GFP”), under the control of the promoter is introduced into a mixed population of cells to be sorted. Mutated forms of GFP that emit more strongly than the native protein, as well as forms of GFP amenable to stable translation in higher vertebrates, are now available and can be used for the same purpose.

The nucleic acid molecule encoding a green fluorescent protein can be deoxyribonucleic acid (“DNA”) or ribonucleic acid (“RNA,” including messenger RNA or mRNA), genomic or recombinant, biologically isolated or synthetic. The DNA molecule can be a cDNA molecule, which is a DNA copy of a messenger RNA (mRNA) encoding the GFP. In one embodiment, the GFP can be from Aequorea victoria (U.S. Pat. No. 5,491,084 to Prasher et al., which is hereby incorporated by reference in its entirety). A plasmid containing cDNA which encodes a green fluorescent protein of Aequorea Victoria is disclosed in U.S. Pat. No. 5,491,084 to Chalfie et al., which is hereby incorporated by reference in its entirety. A mutated form of this GFP (a red-shifted mutant form) designated pRSGFP-C1 is commercially available from Clontech Laboratories, Inc. (Palo Alto, Calif.).

Other suitable marker proteins may be derived from neomycin resistance gene (neomycin phosphotransferase), puromycin resistance gene (puromycin N-acetyl transferase), and hygromycin resistance gene (hygromycin phosphotransferase). When included in plasmid DNA, these genes will make the cells resistant to neomycin, puromycin, and hygromycin, respectively. When cells are cultured in antibiotics, only those with the antiobiotic resistance marker will survive, and those surviving cells can be recovered.

Standard techniques may be used to place the nucleic acid molecule encoding the marker protein under the control of the chosen cell specific promoter. Generally, this involves the use of restriction enzymes and ligation.

The resulting construct, which comprises the nucleic acid molecule encoding the marker protein under the control of the selected promoter (itself a nucleic acid molecule) (with other suitable regulatory elements if desired), is then introduced into a mixed population of cells which are to be sorted. Techniques for introducing the nucleic acid molecules of the construct into the mixed population of cells may involve the use of expression vectors which comprise the nucleic acid molecules. These expression vectors (such as plasmids and viruses) can then be used to introduce the nucleic acid molecule into the mixed population of cells.

Various methods are known in the art for introducing nucleic acid molecules into host cells. These include: 1) microinjection, in which DNA is injected directly into the nucleus of cells through fine glass needles; 2) dextran incubation, in which DNA is incubated with an inert carbohydrate polymer (dextran) to which a positively charged chemical group (DEAE, for diethylaminoethyl) has been coupled (the DNA sticks to the DEAE-dextran via its negatively charged phosphate groups, large DNA-containing particles stick in turn to the surfaces of cells (which are thought to take them in by a process known as endocytosis), and some of the DNA evades destruction in the cytoplasm of the cell and escapes to the nucleus, where it can be transcribed into RNA like any other gene in the cell); 3) calcium phosphate coprecipitation, in which cells efficiently take in DNA in the form of a precipitate with calcium phosphate; 4) electroporation, in which cells are placed in a solution containing DNA and subjected to a brief electrical pulse that causes holes to open transiently in their membranes so that DNA enters through the holes directly into the cytoplasm, bypassing the endocytotic vesicles through which they pass in the DEAE-dextran and calcium phosphate procedures (passage through these vesicles may sometimes destroy or damage DNA); 5) liposomal mediated transformation, in which DNA is incorporated into artificial lipid vesicles, liposomes, which fuse with the cell membrane, delivering their contents directly into the cytoplasm; 6) biolistic transformation, in which DNA is absorbed to the surface of gold particles and fired into cells under high pressure using a ballistic device; 7) naked DNA insertion; and 8) viral-mediated transformation, in which nucleic acid molecules are introduced into cells using viral vectors. Since viral growth depends on the ability to get the viral genome into cells, viruses have devised efficient methods for doing so. These viruses include retroviruses, lentivirus, adenovirus, herpesvirus, and adeno-associated virus.

As indicated, some of these methods of transforming a cell require the use of an intermediate plasmid vector. U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture. The DNA sequences are cloned into the plasmid vector using standard cloning procedures known in the art, as described by Sambrook et al., Molecular Cloning: A Laboratory Manual, 2d Edition, Cold Spring Harbor Laboratory Press, Cold Spring Harbor, N.Y. (1989), which is hereby incorporated by reference in its entirety.

In accordance with one of the above-described methods, the nucleic acid molecule encoding the marker protein is thus introduced into a mixed population of cells. The promoter, which controls expression of the marker protein, however, only functions in the cell of interest. Therefore, the marker protein is only expressed in the cell of interest. When the marker protein is a fluorescent protein, the cells of interest can be identified from among the mixed population of cells by the fluorescence of the fluorescent protein.

Any suitable means of detecting the fluorescent cells can be used. The cells may be identified using epifluorescence optics and can be physically picked up and brought together by Laser Tweezers (Cell Robotics Inc., Albuquerque, N. Mex.). In a preferred embodiment, smooth muscle cells and/or progenitors thereof are separated in bulk through fluorescence activated cell sorting, a method that effectively separates the fluorescent cells from the non-fluorescent cells.

The fluorescent smooth muscle cells or progenitors thereof are then separated from the mixed population of cells by fluorescence activated cell sorting.

The mixed population of cells can be derived from any source where smooth muscle cells are present. Preferred sources of smooth muscle cells include, without limitation, stem cells or progenitors originating in bone marrow or hair follicles. The cells preferably come from an adult human, although embryonic stem cells may also be used.

Another aspect of the present invention is directed to a preparation of isolated smooth muscle cells or progenitors thereof, where the smooth muscle cells or progenitors thereof constitute at least 90% of the preparation. Preferably, these cells are contractile.

In an alternative embodiment, the smooth muscle cells or progenitors thereof constitute at least 95% of the preparation, or at least 99% of the preparation.

A further aspect of the present invention is directed to a method of producing a tissue-engineered vascular vessel. This method involves providing a vessel-forming fibrin mixture containing fibrinogen, thrombin, and the above-described preparation of isolated smooth muscle cells or progenitors thereof. The vessel-forming fibrin mixture is molded into a fibrin gel having a tubular shape. The fibrin gel having a tubular shape is incubated in a medium suitable for growth of the cells under conditions effective to produce a tissue-engineered vascular vessel.

The fibrin gel is derived from a fibrin mixture comprised of fibrinogen, thrombin, and a preparation of isolated smooth muscle cells or progenitors thereof of the present invention. Fibrinogen, thrombin, and isolated smooth muscle cells or progenitors thereof of the fibrin mixture are preferably derived from an autologous source. Preferably, the fibrinogen and thrombin of the fibrin mixture are derived from a patient's blood.

Fibrinogen is a high molecular weight macromolecule (340 kdalton), rodlike in shape, about 50 nm in length, and 3 to 6 nm thick. The central domain contains two pairs of bonding sites, A and B, which are hidden by two pairs of short peptides (fibrinopeptides A and B; FPA and FPB). The polymerization sites A and B are at the ends of the outer domains, where other sites susceptible of enzymatic cross-linking are located. Fibrinogen undergoes polymerization in the presence of thrombin to produce monomeric fibrin. This process involves the production of an intermediate alpha-prothrombin which is lacking one of two fibrinopeptide A molecules, which is then followed rapidly (four times faster), by the formation of alpha-thrombin monomer, lacking both fibrinopeptide A molecules (Ferri et al., Biochemical Pharmacology 62(12):1637-45 (2001), which is hereby incorporated by reference in its entirety). Sites A and B bind to their complimentary sites on other molecules a and b respectively. The aA interaction is responsible for linear aggregation, while the bB interaction is responsible for lateral growth of the fiber. Thrombin cleavage occurs in a particular manner, first cleaving the FPAs to form linear two-stranded, half staggered chains called profibrils. Subsequently, the FPBs are cleaved allowing the fibrils to aggregate side-by-side increasing in diameter. Fibrinogen is naturally cross-linked by components found in plasma, such as protransglutaminase (factor XIII) (Siebenlist et al., Thrombosis & Haemostasis 86(5):1221-8 (2001), which is hereby incorporated by reference in its entirety). This allows for the strengthening of the fibrin gel when in the presence of plasma.

The strength of the fibrin gel adhesive component may depend on the final concentration of fibrinogen. Higher fibrinogen concentrations can be achieved by increasing the mixing ratio of the typical 1:1 (thrombin:fibrinogen) mixture of the present invention to a 1:5 mixture.

The cells in the vessel-forming fibrin mixture are preferably at a concentration within the vessel-forming fibrin mixture of about 1 to 4×10⁶ cells/ml.

The vessel-forming fibrin mixture of the present invention is molded into a fibrin gel having a tubular shape. The compaction of fibrin gels is a process poorly understood. If compaction were to occur in an unconstrained system such as, in a well after being released from the surface, the cells and fibrin fibers show very little organization or alignment. However, when cells compact a fibrin gel in the presence of an appropriate mechanical constraint, a circumferential alignment of fibrils and cells results, which resembles that of the vascular media (Weinberg and Bell, Science 231(4736):397-400 (1986); L'Heureux et al., Journal of Vascular Surgery 17(3):499-509 (1993), which are hereby incorporated by reference in their entirety). This alignment characteristic is very important in the development of functionality. Mechanical function is dependent on structure, interactions of cells, and extracellular matrix (alignment), equally to that of composition. Function is also important in the remodeling of the tissue-engineered vasculature vessels. Their structure-function relationship provides a template for the vessel as remodeling occurs.

Molding of the fibrin mixture is preferably carried out in a silastic tube with an inner mandrel. Fibrin gel has the ability to become aligned near a surface as the gel is formed or within the gel as it compacts due to traction exerted by entrapped cells (Tranquillo, Biochem. Soc. Symp. 65:27-42 (1999), which is hereby incorporated by reference in its entirety). The use of a central mandrel during gelation increases circumferential alignment of the smooth muscle cells as well as the matrix. The use of a mandrel also provides a large stress on the smooth muscle cells which induces secretion and accumulation of extracellular matrix that enhances the stiffening component of the construct (Barocas et al., J. Biomech. Eng. 120(5):660-6 (1998), which is hereby incorporated by reference in its entirety).

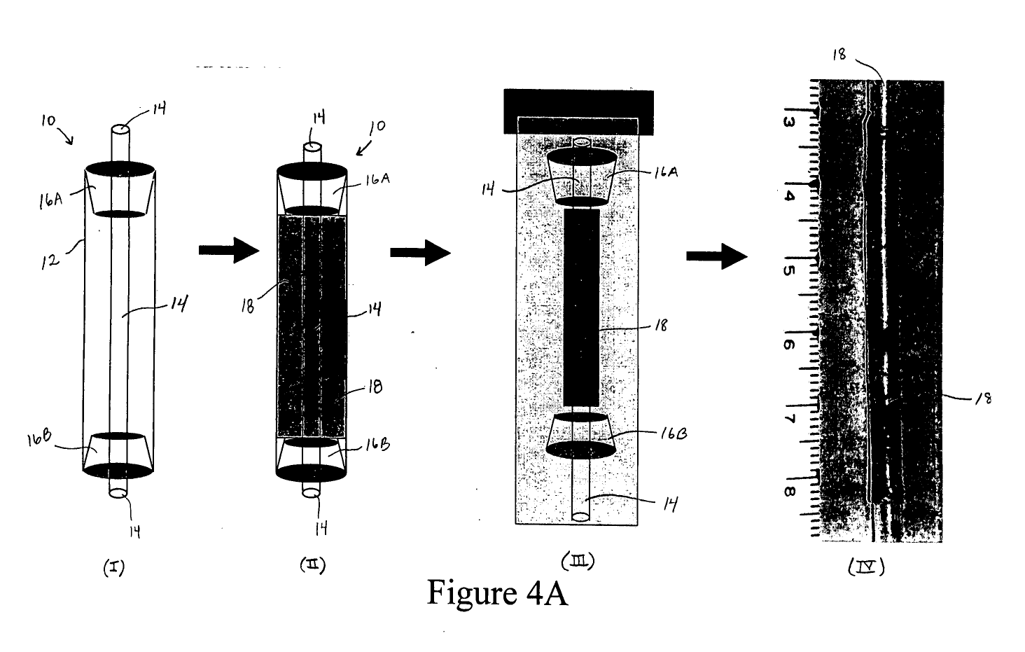

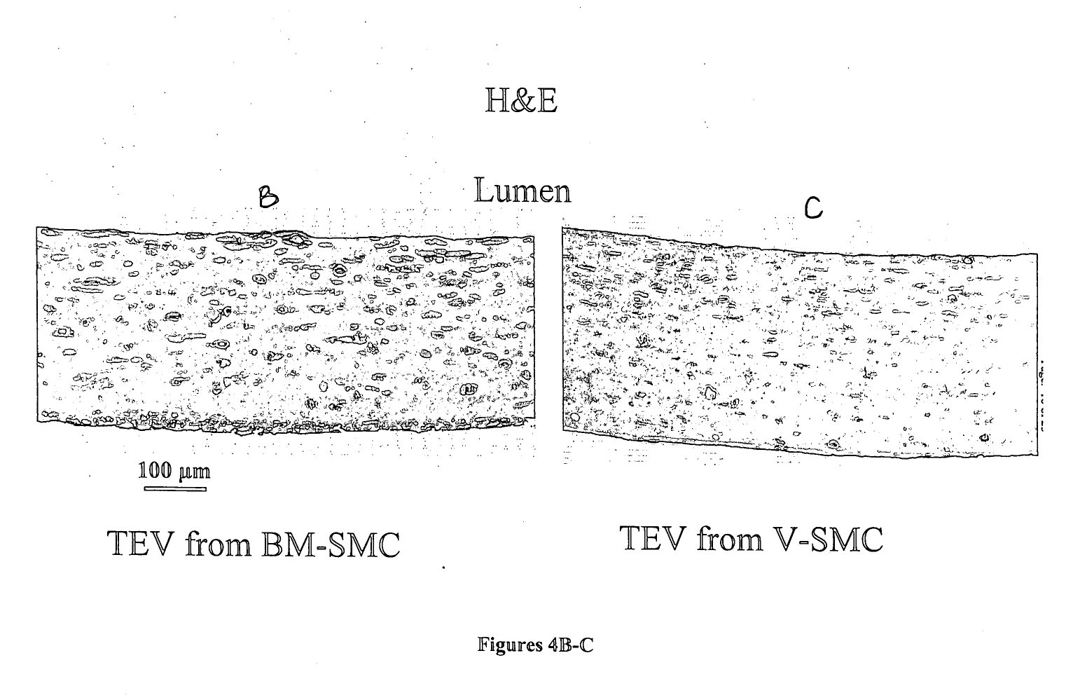

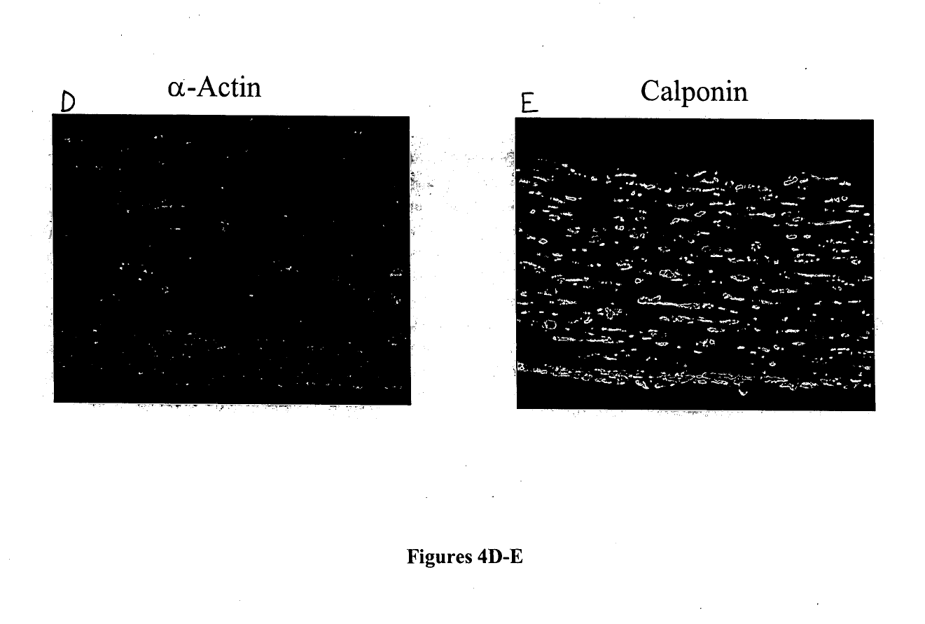

FIG. 4A illustrates one embodiment of the method of producing a tissue-engineered vascular vessel of the present invention. As shown in illustration (I) of FIG. 4A, vessel forming device 10 has silastic tube 12, which is occupied by inner mandrel 14. Stoppers 16A-B can be fitted into both ends of silastic tube 12. Illustration (II) shows silastic tube 12, which has been filled with vessel-forming fibrin mixture 18 around inner mandrel 14. Illustration (III) shows compaction of vessel-forming fibrin mixture 18 around inner mandrel 14. A photograph of tissue engineered vessel 18, made by the process illustrated in FIG. 4A, is shown in illustration (IV).

During development of the tissue-engineered vasculature of the present invention, it may be desirable to pulse the vessel constructs to modulate growth, development, and structure and/or function of the vessels. When the fibrin vessel constructs are pulsed, there is an inhibition of longitudinal compaction of the construct. In the case of adding a continuous rhythmic pulsation, an increase in cellular alignment perpendicular to the applied force may be achieved. The increased radial alignment created from pulsation may be the limiting factor of the longitudinal compaction.

Pulsing may be achieved by applying force directly to the inner lumen of the tissue-engineered vessel constructs. For example, a roller pump may be used to pass liquid through the inner lumen of the vessels in a pulsating manner. Alternatively, the inner mandrel used in molding the vessel constructs may be connected to a pneumatic pulsation device. In some instances pulsation may have a desirable effect on the structure and/or function of the vessel. In other instances, pulsation may have a detrimental effect on the desired characteristics (structure and/or function) of the vessel.

After incubation of the fibrin gel, it is preferable to grow the cells of the fibrin mixture in a medium suitable for growth. The optimization of the fibrin gel vascular construct includes a multitude of growth factors that can be used to further development and function. In particular, high serum medias as well as keratinocyte growth factor (“KGF”) demonstrate an enhanced development of the fibrin gel vascular vessel construct. Also, literature cites the use of many other growth factors that have stimulated cell growth, function, and behavior when used with fibrin and other gels.

A suitable medium of the present invention is comprised of M199, 1% penicillin/streptomycin, 2 mM L-glutamine, 0.25% fungizone, and 15 mM HEPES. A growth additive may also be added to the medium suitable for growth. A suitable growth additive is comprised of 50 μg/ml ascorbic acid, 10-20% FBS, 10-20 μg/ml aprotinin or 0.5-2.0 mg/ml EACA, 2 μg/ml insulin, 5 ng/ml TGFβ1, and 0.01 U/ml plasmin. In addition, a growth hormone may be included in the growth additive. Suitable growth hormones include, VEGF, b-FGF, PDGF, and KGF. Preferably, the growth medium is changed every 2-3 days.

Endothelial cells may be seeded to the interior of the tissue-engineered vascular vessel by removing the inner mandrel and seeding the cells to the interior lumen of the vessel. Cells may also be added to the outer surface of the vessels during molding. Suitable cells to be seeded to the outer surface of the vessel include, in a preferred embodiment, fibroblasts. Alternatively, specific organ cells may be seeded to the outer surface of the tissue-engineered vascular vessel of the present invention.

The tissue-engineered vascular vessel of the present invention may also be comprised of a fibrin gel scaffold combined with a porous scaffold to enhance vascular grafting. When the same fibrin gel containing a uniform distribution of cells is used in conjunction with other highly porous scaffold materials, there may be many synergistic benefits of this composite fibrin gel scaffold. There are all the benefits of the fibrin gel plus the addition of early interim strength and early incorporation of other factors that may typically not be produced until later in development (elastin). Thus, the fibrin gel of the present invention can be used with any porous scaffold, such as decellularized elastin or poly lactic-glycolic acid (“PLGA”) to further enhance the benefits and applicability of the fibrin gel vascular grafts. A preferable porous scaffold to be combined with fibrin gel to enhance vascular grafting is decellularized elastin. Another preferable porous scaffold to be combined with fibrin gel to enhance vascular grafting is PLGA.

Smooth muscle cells are known to rapidly degrade fibrin via secretion of proteases. Thus, it is desirable to prevent this degradation during the development of the tissue-engineered vessel of the present invention. Degradation of fibrin in the vessel of the present invention can be controlled through the use of protease inhibitors. A suitable protease inhibitor of the present invention is aprotinin. In a preferred embodiment of the present invention, 0 to 200 μg/ml of aprotinin is added to the fibrin mixture to modulate fibrin degradation. Preferably, about 20 μg/ml of aprotinin is added to the fibrin mixture to modulate fibrin degradation.

Aprotinin has the ability to slow or stop fibrinolysis. Particularly, aprotinin acts as an inhibitor of trypsin, plasmin, and kallikrein by forming reversible enzyme-inhibitor complexes (Ye et al., European Journal of Cardio-Thoracic Surgery 17(5):587-91 (2000), which is hereby incorporated by reference in its entirety). ε-aminocaproic acid (“EACA”), another suitable protease inhibitor of the present invention, binds plasmin to inhibit fibrinolysis (Grassl et al., J. Biomed. Mater. Res. 60(4):607-12 (2002), which is hereby incorporated by reference in its entirety). Supplementation with a protease inhibitor (EACA or aprotinin) to control the rate of degradation, may have a modulating effect on collagen synthesis, which is dependent on the rate of degradation (Grassl et al., J. Biomed. Mater. Res. 60(4):607-12 (2002), which is hereby incorporated by reference in its entirety). As collagen is produced, more than half appears in the medium as an aggregate with the balance retained in the matrix (Grassl et al., J. Biomed. Mater. Res. 60(4):607-12 (2002), which is hereby incorporated by reference in its entirety).

Total weight of the fibrin vessel constructs of the present invention can be affected by the amount of aprotinin added to the medium. This is evident from the increase in weight of the total vessel construct as greater amounts of aprotinin are added. However, vessel weight is not controlled totally by the addition of aprotinin, because it has been observed that non-pulsed vessel weight plateaus, while pulsed vessel weight continues to rise with increasing aprotinin. Thus, there appears to be a balance between secreted proteases, extracellular matrix secretion, and the added aprotinin in combination with pulsation. Further optimization of overall development of the tissue-engineered vascular vessels of the present invention can be obtained by adjusting the amount and degree of pulsation during development and the concentration of aprotinin.

Yet another aspect of the present invention is directed to a tissue-engineered vascular vessel containing a gelled fibrin mixture having fibrinogen, thrombin, and the preparation of isolated smooth muscle cells or progenitors thereof as described above. The gelled fibrin mixture has a tubular shape.

The tissue-engineered vascular vessel of the present invention is suitable as an in vivo vascular graft. In vivo vascular grafts of the tissue-engineered vascular vessels of the present invention may be made in animals. In a preferred embodiment, the vessel is used as a vein graft in a human being.

The mechanical properties of the tissue-engineered vasculature of the present invention are of major importance when determining development or appropriateness of the vessels. In particular, properties such as collagen content, cell proliferation, cell density, reactivity, and vessel constriction determine how the vessels function physically in terms of compliance and strength. It is desirable that the tissue-engineered vascular vessels of the present invention demonstrate a remarkable development in both compliance and strength in a short period of time.

It has been shown that factors such as TGFβ, insulin, plasmin, and time contribute to increasing collagen content in fibrin gels (Neidert et al., Biomaterials 23(17):3717-31 (2002), which is hereby incorporated by reference in its entirety).

Yet a further aspect of the present invention is directed to a method of producing a tissue-engineered vascular vessel for a particular patient. This method involves providing a vessel-forming fibrin mixture containing fibrinogen, thrombin, and the preparation of isolated smooth muscle cells or progenitors described above, at least one of which is autologous to the patient. The vessel-forming fibrin mixture is molded into a fibrin gel having a tubular shape. The fibrin gel having a tubular shape is incubated in a medium suitable for growth of the cells under conditions effective to produce a tissue-engineered vascular vessel for a particular patient. The tissue-engineered vascular vessel is implanted into the particular patient.

Preferably, the fibrinogen and the cells are autologous, i.e., derived from the patient. More preferably, fibrinogen is isolated from the patient's blood.

These aspects of the present invention are further illustrated by the examples below.

### EXAMPLES

The following examples are provided to illustrate embodiments of the present invention, but they are by no means intended to limit its scope.

### Example 1

### Cloning of SMαA Promoter

Rat SMαA promoter DNA was amplified from rat genomic DNA (Clontech, Mountain View, Calif.) using high fidelity PCR with:

forward primer: ACGGTCCTTAAGCATGATAT (SEQ ID NO:1); and

reverse primer: CTTACCCTGATGGCGACTGGCTGG (SEQ ID NO:2) (Hu et al., “Smad3 Mediates Transforming Growth Factor-Beta-Induced Alpha-Smooth Muscle Actin Expression,” Am. J. Respir. Cell. Mol. Biol. 29(3 Pt 1):397-404 (2003), which is hereby incorporated by reference in its entirety). The PCR reaction was carried out with denaturation for 30 s at 94° C.; annealing for 30 s at 55° C.; and extension for 90 s at 72° C. The PCR product was cloned into the pCR2.1 TOPO vector (Invitrogen, Carlsbad, Calif.) and was subsequently excised with XhoI and BamHI and subcloned into the same sites of the promoterless EGFP reporter vector (pEGFP-1, Clontech).

### Example 2

### Isolation of Bone Marrow-Derived Smooth Muscle Cells

BM-MNC from a newborn lamb were separated from a bone marrow aspirate using histopaque (1.077 g/ml) density-gradient centrifugation (Sigma, St. Louis, Mo.). BM-MNC cells were plated on 6 well plates and maintained in DMEM (Gibco, Grand Island, N.Y.) containing 10% FBS (Gibco).

When BM-MNC reached 70% confluence, the cells were transfected with SMαA-EGFP plasmid DNA using lipofectamine (Invitrogen, Carlsbad, Calif.) as per the manufacturer's instructions. Briefly, BM-MNC were washed three times with serum-free, antibiotic-free DMEM. Lipofectamine:DNA (8 μ1:2 μg) complex was prepared in 0.2 ml serum-free, antibiotic-free DMEM and incubated at room temperature for 30-45 minutes. For transfection, 0.8 ml of serum-free DMEM was added into the lipid:DNA solution and overlaid on BM-MNC for 5 hr at 37° C. After incubation, the transfection mixture was removed and replaced with DMEM containing 10% FBS (Invitrogen). The next day, the culture medium was replenished again and at 72 hr post-transfection, EGFP-expressing cells were observed by fluorescence microscopy and sorted by FACS.

### Example 3

### Isolation of Bone Marrow-Derived Endothelial Cells

BM-MNC were separated from a bone marrow aspirate as described above, seeded onto a 100 mm tissue culture dish coated with 20 ng/ml of human fibronectin (Calbiochem, La Jolla, Calif.), and cultured in DMEM containing 20% of FBS at 10% CO₂, 37° C. The next day, non-adherent cells were transferred onto a new fibronectin-coated plate and cultured in the same medium for 24 hr before the non-adherent fraction was transferred again to a third fibronectin-coated plate. The adherent cells were cultured until cell colonies were large enough to be picked. At that time, individual colonies containing cells that displayed cobblestone morphology were isolated using trypsin-soaked cloning disks (Scienceware, Santa Ana, Calif.), and transferred into one well of 6-well plate each in the same medium. The next day the medium was replaced by human endothelial-SFM basal growth medium (“EGM”) supplemented with 10 μg/ml of human plasma fibronectin (Calbiochem), 10 ng/ml of epidermal growth factor (BD Biosciences, Bedford, Mass.), 20 ng/ml of human fibroblast growth factor (BD Biosciences). The cells were cultured in EGM with all supplements and sub-cultured when they reached 80% confluence. These cells were termed BM-EC and used for experiments between 4-6 passages.

### Example 4

### Immunohistochemistry

For immunostaining, BM-SMC were fixed with 4% paraformaldehyde for 10 min at room temperature, then permeabilized with 0.1% triton®-X-100 (Fisher Scientific) for 1 hr, blocked with 1% BSA in PBS for 30 min and incubated with monoclonal antibodies overnight at 4° C. The following antibodies were used: mouse monoclonal anti-human smooth muscle actin (1:100 dilution; SeroTec, Oxford, UK), anti-human smooth muscle calponin (1:100 dilution; DakoCytomation, Carpinteria, Calif.) in PBS containing 1% BSA and 0.01% triton®-X-100. The cells were washed three times and incubated with Alexa Fluor594 goat anti-mouse IgG (1:200 dilution; Molecular Probes, Eugene, Oreg.) for 1 hr at room temperature.

BM-EC were fixed with cold acetone at −30C for 10 minute, then blocked with 10% goat serum in PBS for 1 hr and incubated with primary antibodies for 2 hr at room temperature. The following antibodies were used: mouse anti-ovine CD31 conjugated with FITC (1:10 dilution; Serotec), rabbit anti-human CD144 conjugated with FITC(1:10 dilution; Serotec), polyclonal rabbit anti-human von Willebrand Factor (1:10 dilution; DakoCytomation). For vWF staining the cells were washed three times and incubated with Alexa Fluor® 488 goat anti-rabbit IgG (1;100 dilution; Molecular Probes) for 1 hr at room temperature.

After antibody staining, cells (BM-SMC or BM-EC) were washed three times and incubated with Hoechst 33325 (1:400 dilution; Molecular Probes, Eugene, Oreg.) for 10 min at room temperature, followed by three more washes with PBS and mounting in aqueous medium (Gel/Mount; Biomeda). Stained cells were visualized with an inverted fluorescence microscope (Diaphot-TMD; Nikon Instruments, Melville, N.Y.). Images were acquired at 40× magnification using a Retiga 1300 digital camera (QImaging, Burnaby, BC, Canada) and analyzed using QCapture2 software, version 1.1 (QImaging).

### Example 5

### Western Blots

BM-SMC or V-SMC were washed with ice-cold PBS, lysed with 1 ml of RIPA Buffer (150 mM NaCl, 10 mM Tris PH 7.2, 0.1% SDS, 1.0% Triton®-X-100, 1% deoxycholate, 5 mM EDTA) containing a cocktail of protease inhibitor (Roche Diagnostics GmbH Mannheim, Germany). Cell lysates were sonicated, diluted with SDS sample buffer (after dilution the composition of the sample buffer was: 62.5 mM Tris-HCl, 10% v/v glycerol, 2% w/v SDS, 0.01% bromophenol blue, 41.6 mM DTT), and heated at 95° C. for 5 min. After centrifugation at 12,000 rpm for 5 min, the lysates were separated by SDS-Page (12%) and transferred to nitrocellulose membrane (transfer buffer: 25 mM Tris-HCl, pH 8.3, 192 mM glycine, 20% (v/v) methanol, 0.1% (w/v) SDS) for 1 hr using an electrophoretic transfer cell (Mini Trans-Blot®; BioRad Laboratories, Hercules, Calif.).

Membranes were blocked with blocking buffer containing 5% (w/v) nonfat dry milk in TBS-Tween (20 mM Tris-HCl, pH 7.2-7.4, 150 mM NaCl, 0.1% (v/v) Tween 20) on a rocker platform for 2 hr at room temperature. The membranes were washed three times in TBS-Tween and were incubated with primary antibodies in blocking buffer overnight at 4° C. on a rocker plate for continuous mixing. The following antibodies were used: mouse anti human smooth muscle α-actin (1:100 dilution; SeroTec) and mouse anti-human smooth muscle calponin (1:100 dilution; DakoCytomation) in TBS-Tween. After incubation the membranes were washed five times for a minimum of 5 min each in TBS-Tween and incubated with horseradish peroxidase-conjugated goat anti-mouse IgG (1:5,000 dilution; Cell Signaling Technology, Danvers, Mass.) in blocking buffer for 1 hr at room temperature. The membrane was washed five times with TBS-Tween and the protein bands were detected using chemiluminescence (LumiGLO; KPL, Gaithersburg, Md.) as per manufacturer's instructions. Western blots using BM-TEV lysates were performed as using the same protocol.

### Example 6

### Tissue Engineering of Small Diameter Blood Vessels

TEV from BM-SMC were prepared as described previously (Swartz et al., “Engineering of Fibrin-Based Functional and Implantable Small-Diameter Blood Vessels,” Am. J. Physiol. Heart Circ. Physiol. 288(3):H1451-1460 (2005); Yao et al., “Fibrin-Based Tissue-Engineered Blood Vessels: Differential Effects of Biomaterial and Culture Parameters on Mechanical Strength and Vascular Reactivity,” Tissue Eng. 11(7-8):991-1003 (2005), which are hereby incorporated by reference in their entirety). Briefly, BM-SMC were suspended in thrombin in the presence of calcium. The thrombin containing BM-SMC were mixed with fibrinogen at a ratio of 1:4, poured into a plastic mold (1 cm×6 cm) surrounding a silastic tube with diameter of 4.0 mm and polymerized within 5-10 seconds. The final concentration of each component was: 2.5 mg/ml fibrinogen, 2.5 mM calcium and 2.5 U/ml of thrombin. The final cell density of BM-SMC was 1×10⁶ cells/ml gel. After 1 hr incubation in a CO₂ incubator, the BM-TEV were detached from the walls, removed from the plastic tube, and transferred into a 50 ml conical tube, where they were incubated in 40 ml of DMEM medium containing 25 mM Hepes, 20% FBS, and 300 μM ascorbic acid phosphate. The next day the medium was supplemented with 2 μg/ml insulin, 5 ng/ml TGF-β1, and 20 μg/ml aprotinin. Thereafter, cell culture medium was replenished every three days. After two weeks in culture, the cylindrical tissues were removed from the mandrel and BM-EC were seeded in the lumen at 6×10⁵ cells/cm² of luminal area. The cells were allowed to adhere for 4 hr under continuous rotation of the cylindrical constructs to ensure uniform seeding. Subsequently, the tissues were cultured in M199 containing 20% FBS for another 10 days before implantation.

### Example 7

### Vasoreactivity and Mechanical Strength of BM-TEV

After two weeks in culture, BM-TEV were released from the mandrel and cut in 3-4 mm segments, mounted on two stainless hooks in an isolated tissue bath, and incubated in Krebs-Ringer solution. The tissues were continuously bubbled with 94% OB₂, 6% CO₂ to obtain a pH of 7.4, a pCO₂ of 38 mmHg, and a PO₂>500 mmHg at 37° C. Each construct was mounted on stainless steel hooks through the lumen; one was fixed and the other one was connected to a force transducer. Tissues were equilibrated at a basal tension of 1.0 g and constant length for 30-60 min. After equilibration, potassium chloride (KCl, 118 mM) or norepinephrine (NE, 10⁻⁶ M) was added to the tissue bath and isometric contraction was recorded by a PowerLab data acquisition unit and analyzed by Chart5 software (ADInstruments, Colorado Springs, Colo.).

Tissue segments were mounted on the force transducer and stretched incrementally until they broke, yielding the break tension and break length of the tissue. The initial tissue length corresponds to the length under a passive tension of 1.0 g. Broken constructs were dehydrated with series of ethanol washes, air dried, and weighted. The force was normalized by the dry weight of each construct and expressed in units of Newton per gram tissue weight (N/g dry weight). Linear modulus was calculated as the slope of the linear part of the length-tension curve. Toughness was calculated by numerically integrating the area under the length-tension curve after fitting the curve by the method of least squares, using Maple 9.0 software (Waterloo Maple, Waterloo, ON, Canada). Toughness was expressed in units of millimeters×Newton/gram tissue weight (mm N/g dry weight).

### Example 8

### Histology and Immunohistochemistry

Tissue samples were fixed in 10% buffered formalin, dehydrated through graded concentrations of ethanol and Hemo-De (Scientific Safety Solvents, Keller, Tex.) and embedded in paraffin. Tissue morphology and collagen synthesis were evaluated by staining 5 μm paraffin sections with H&E and Masson's trichrome, H respectively, as described previously (Yao et al., “Fibrin-Based Tissue-Engineered Blood Vessels: Differential Effects of Biomaterial and Culture Parameters on Mechanical Strength and Vascular Reactivity,” Tissue Eng. 11(7-8):991-1003 (2005); Geer et al., “In Vivo Model of Wound Healing Based on Transplanted Tissue-Engineered Skin,” Tissue Eng. 10(7):1006-1017 (2004); Geer et al., “Biomimetic Delivery of Keratinocyte Growth Factor Upon Cellular Demand for Accelerated Wound Healing In Vitro and In Vivo,” Am. J. Pathol. 167(6):1575-1586 (2005), which are hereby incorporated by reference in their entirety). Images of tissue sections were acquired on an inverted microscope (Diaphot-TMD; Nikon Instruments, Melville, N.Y.) using a Retiga 1300 digital Camera (QImaging, Burnaby, BC, Canada) and QCapture2 software, version 1.1 (QImaging).

For immunohistochemistry, tissue sections were deparaffinized and incubated in a tissue section retriever (PickCell Laboratories BV, Amsterdam, NL) for antigen unmasking. Tissue sections were permeabilized with 0.1% triton®-X-100 for 1 hr, blocked with 1% BSA in PBS for 30 min and incubated with monoclonal antibodies overnight at 4° C. The following antibodies were used: mouse monoclonal anti-human smooth muscle actin (1:100 dilution; SeroTec), anti-human smooth muscle calponin (1:100 dilution; DakoCytomation) in PBS containing 1% BSA and 0.01% triton X-100. The tissue sections were washed three times and incubated with Alexa Fluor488 or Alexa Fluor594 goat anti-mouse IgG (1:100 dilution; Molecular Probes) for 1 hr at room temperature. Images of tissue sections were acquired using an inverted microscope (Diaphot-TMD; Nikon Instruments) and a Retiga 1300 digital camera (QImaging, Burnaby, BC, Canada).

### Example 9

### BM-TEV Implantation

BM-TEV were implanted into the jugular vein of 8-week old lambs as described previously (Swartz et al., “Engineering of Fibrin-Based Functional and Implantable Small-Diameter Blood Vessels,” Am. J. Physiol. Heart Circ. Physiol. 288(3):H1451-1460 (2005), which is hereby incorporated by reference in its entirety). Briefly, 8 week-old dorset cross castrate males (˜25 kg) were fasted 24 hr prior to surgery. Anesthesia was induced with sodium pentathol (50 mg/animal) and maintained with 1.5-2.0% isoflurane through a 6.0 mm endotracheal tube using a positive pressure ventilator and 100% oxygen. The left external jugular vein was exposed through a longitudinal 8 cm incision. After tying small collateral vessels, 3,000 units of heparin sulfate were administered and the proximal and distal ends of the implantation site were clamped.

The external jugular vein was transected and a 1.0-1.5 cm segment of the TEV was sutured into place using continuous running 8-0 proline cardiovascular double armed monofilament suture (Ethicon, Johnson and Johnson, Somerville, N.J.). The vascular clamp was slowly removed and flow was resumed through the TEV graft. A radiopaque tie was loosely secured at the caudal end of the TEV to mark the location of the graft. The incision was closed using 2-0 vicryl in layers (facia and skin). The animal was recovered and monitored daily for adverse affects. Angiograms were performed between 6 and 8 weeks post grafting. At that time the animals were euthanized using 10 ml concentrated sodium barbiturate (Fatal Plus; Vortech Pharmaceuticals, Dearborn, Mich.). TEV grafts were removed along with intact caudal and cephalic native vessel. Tissue segments were processed for histology and immunohistochemistry. All procedures and protocols in this study were approved by the Laboratory Animal Care Committee of the State University of New York at Buffalo.

### Example 10

### Isolation, Characterization, and Expansion of Functional Smooth Muscle Cells from Bone Marrow

Data were expressed as mean±standard deviation and statistical significance (defined as p<0.05) was determined using Student's t-test.

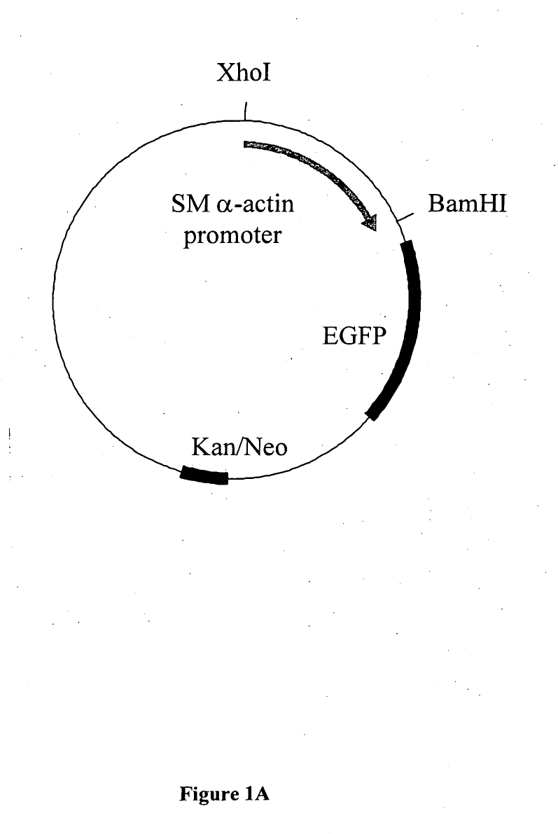

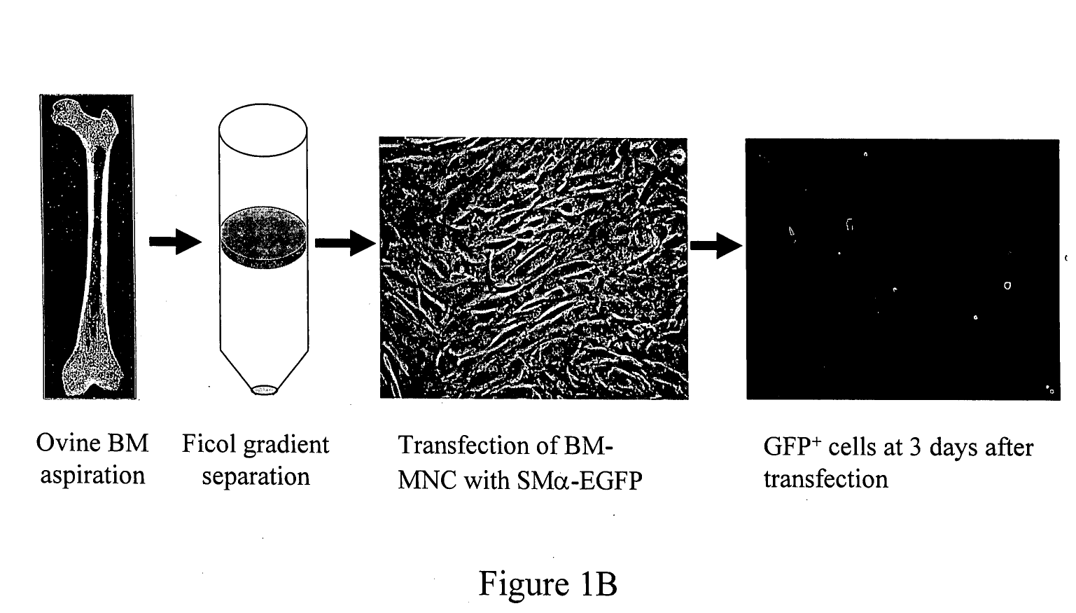

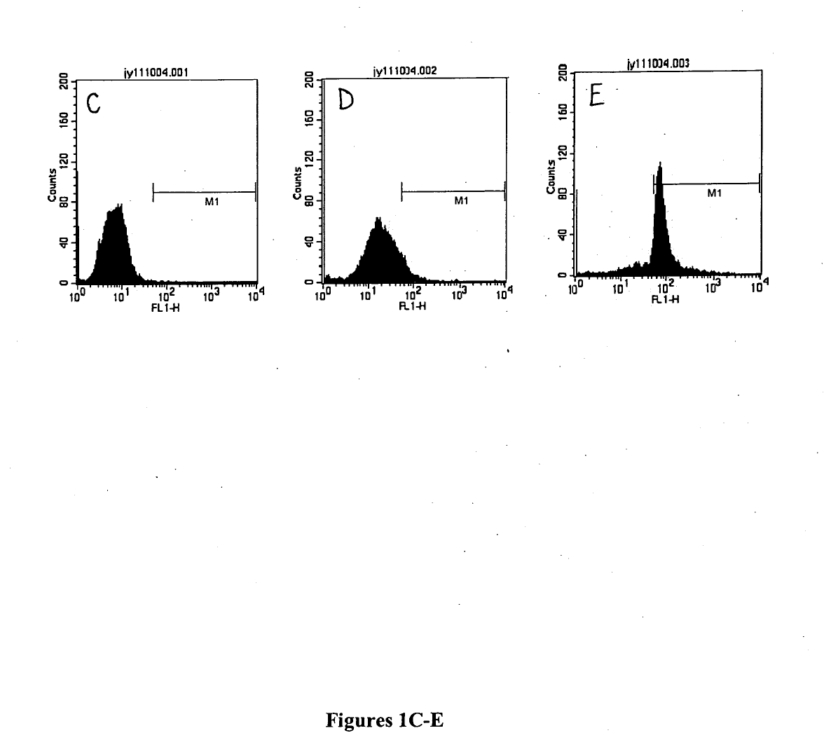

To ensure isolation of smooth muscle cells from bone marrow mononuclear cells with no contamination from any other cell type, the method of the present invention is based on expression of green fluorescence protein from the SMαA promoter. The rat SMαA promoter (Accession Number S76011) was PCR-amplified from rat genomic DNA and cloned into a promoterless vector encoding for EGFP (FIG. 1A). Bone marrow was harvested from newborn lambs and mononuclear cells were isolated by density gradient centrifugation using histopaque and grown in DMEM medium with 10% FBS. Non-adherent cells were discarded, adherent mononuclear cells were transfected with the SMαA-EGFP plasmid, and EGFP+cells were subsequently sorted using fluorescence activated cell sorting (FIG. 1B and FIGS. 1C-E).

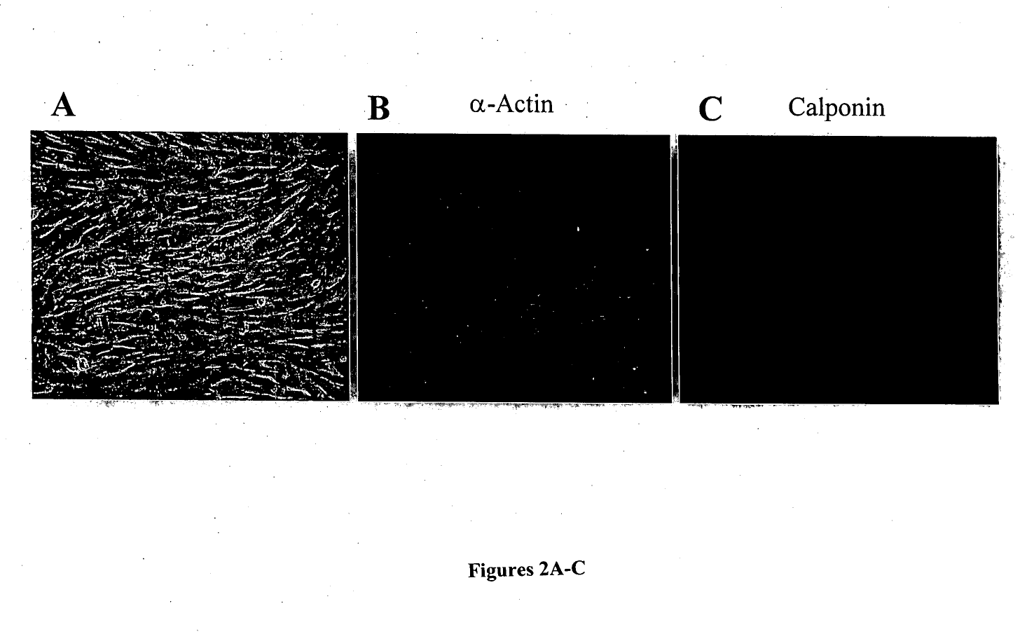

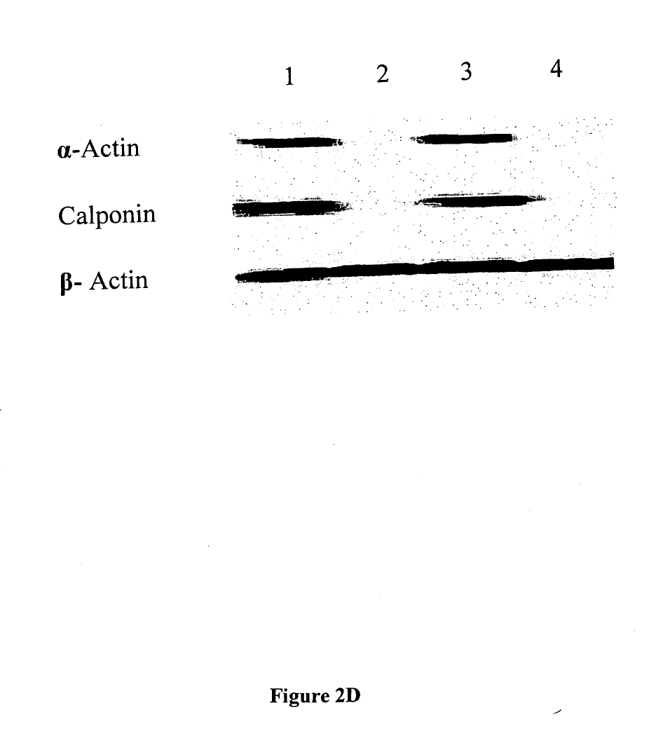

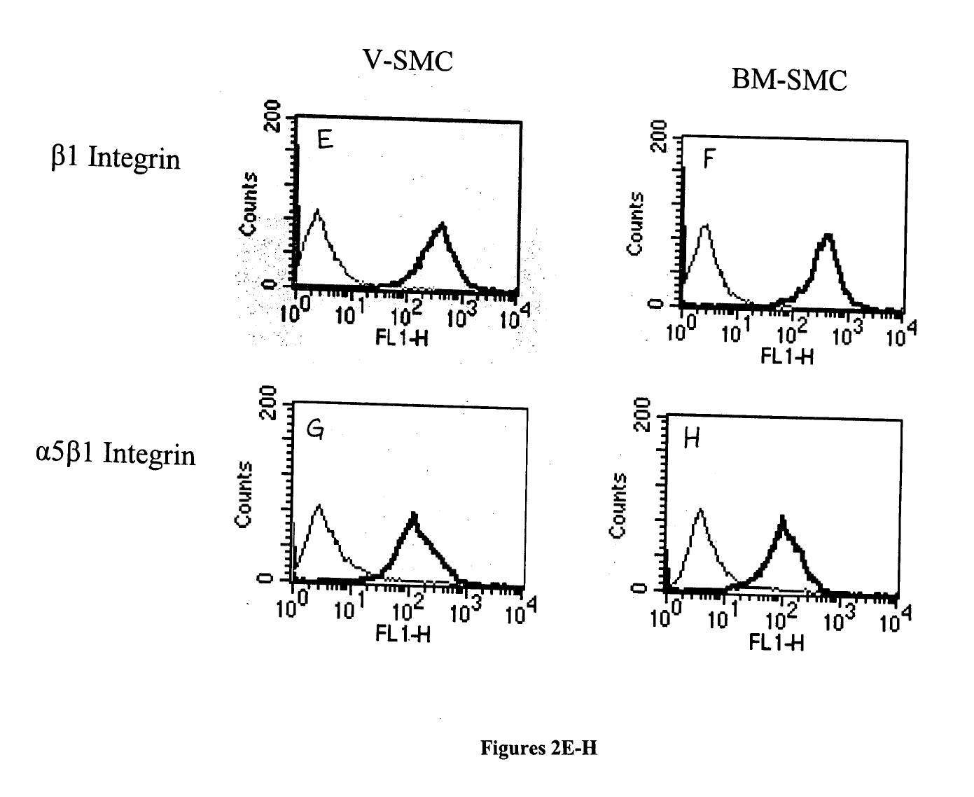

The sorted cells (BM-SMC) displayed SMC-like morphology (i.e. they were elongated, spindle-shaped, and contained a well-developed actin stress fiber network) (FIG. 2A). In addition, immunostaining showed that BM-SMC stained strongly for anti-smooth muscle α-actin and calponin (FIG. 2B and FIG. 2C). In addition, Western Blots showed that BM-SMC expressed smooth muscle α-actin and calponin to a similar extent as V-SMC, while as expected, ovine vascular endothelial cells and human epidermal keratinocytes expressed neither protein (FIG. 2D). Flow cytometry showed that BM-SMC expressed high amounts of integrin α5 and β1 on their surface, similar to V-SMC (FIGS. 2E-H). This result is in agreement with a previous study that showed high integrin expression in smooth muscle progenitor cells from bone marrow and peripheral blood (Simper et al., “Smooth Muscle Progenitor Cells In Human Blood,” Circulation 106(10):1199-1204 (2002), which is hereby incorporated by reference in its entirety).

Notably, BM-SMC could be sub-cultured repeatedly with no apparent loss of proliferative potential even after 12 passages. In contrast, mature V-SMC terminally differentiated and stopped proliferating after 5-6 passages, suggesting that BM-SMC have a higher proliferation potential and may be a better cell source for cardiovascular tissue engineering.

### Example 11

### Isolation, Characterization, and Expansion of Endothelial Progenitor Cells from Bone Marrow

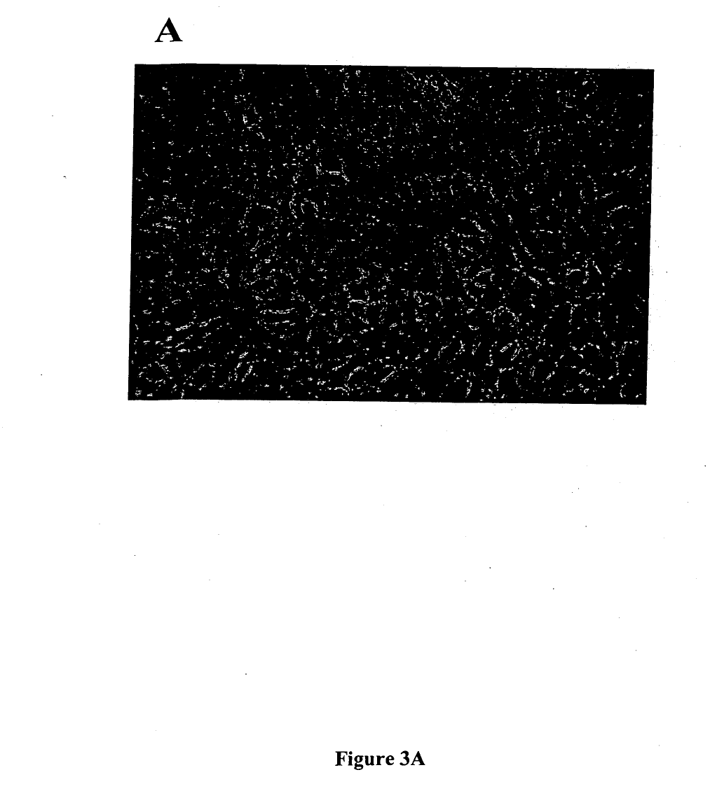

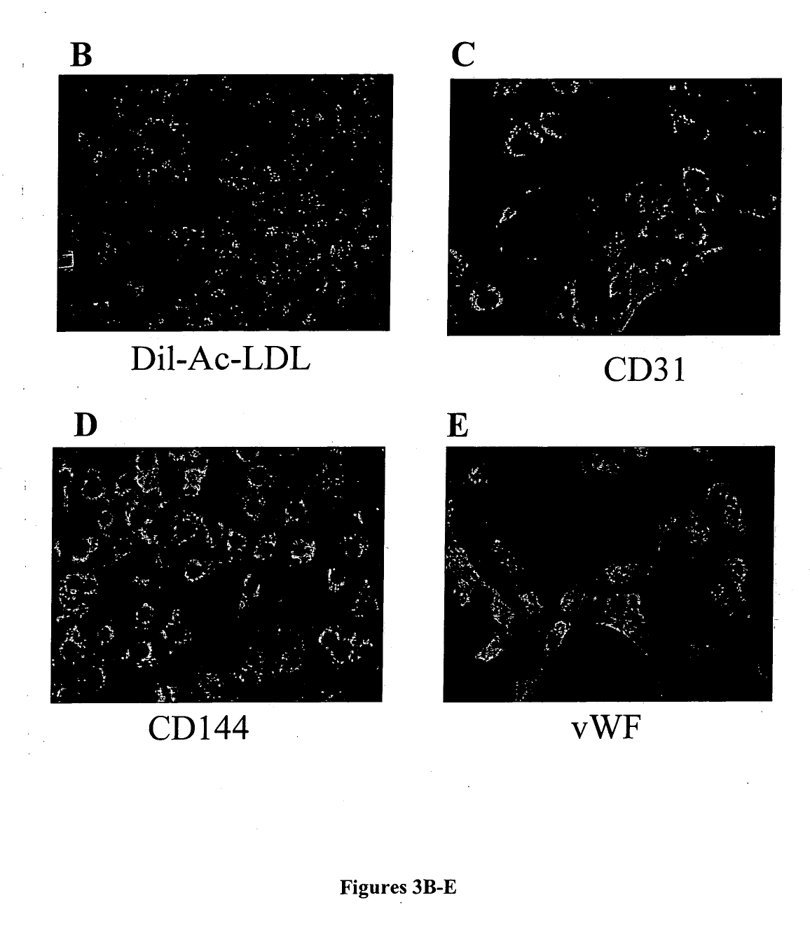

Endothelial cells were isolated from bone marrow based on differential adhesion of BM-MNC (FIG. 3A) on fibronectin as published previously (Shi et al., “Evidence for Circulating Bone Marrow-Derived Endothelial Cells,” Blood 92(2):362-367 (1998); Peichev et al., “Expression of VEGFR-2 and AC133 by Circulating Human CD34(+) Cells Identifies a Population of Functional Endothelial Precursors,” Blood 95(3):952-958 (2000); Kaushal et al., “Functional Small-Diameter Neovessels Created Using Endothelial Progenitor Cells Expanded Ex Vivo,” Nat. Med. 7(9):1035-1040 (2001), which are hereby incorporated by reference in their entirety). When grown to confluence in EGM medium supplemented with fibronectin, EGF and bFGF bone marrow-derived endothelial cells (BM-EC) exhibited cobblestone morphology (FIG. 3B) and stained positively for DiI-Ac-LDL, CD31, CD144 and vWF (FIGS. 3B-E). As expected, BM-SMC did not stain for any of these proteins.

### Example 12

### BM-SMC Can Be Used to Engineer Functional Bioengineered Blood Vessels

Next, it was examined whether BM-SMC could be used to prepare cylindrical tissue engineered blood vessels (BM-TEV). To this end, BM-SMC were embedded in fibrin hydrogels that were polymerized around 4-mm mandrels and cultured in the presence of insulin, TGF-β1, and aprotinin, a combination that was previously shown to promote extracellular matrix synthesis, mechanical strength, and vascular reactivity of the TEV generated from V-SMC (Yao et al., “Fibrin-Based Tissue-Engineered Blood Vessels: Differential Effects of Biomaterial and Culture Parameters on Mechanical Strength and Vascular Reactivity,” Tissue Eng. 11 (7-8):991-1003 (2005); Neidert et al., “Enhanced Fibrin Remodeling In Vitro With TGF-Beta 1, Insulin and Plasmin for Improved Tissue-Equivalents,” Biomaterials 23(17)3717-3731 (2002), which are hereby incorporated by reference in their entirety).

Similar to V-SMC, TEV prepared with BM-SMC compacted fibrin hydrogels to approximately 5% of their original volume within 3 days in culture (FIG. 4A), indicating that these cells had developed the ability to generate force. After two weeks in culture the tissues were removed from the mandrel and processed for histology and immunohistochemistry. Similar to V-SMC (FIG. 4B), BM-SMC (FIG. 4C) distributed uniformly in the fibrin hydrogel and stained positive for smooth muscle α-actin (FIG. 4D) and calponin (FIG. 4E). These results were confirmed by western blots.

### Example 13

### BM-TEV Display Significant Vascular Reactivity

The defining property of mature SMC is their ability to contract and generate force in response to vasoactive agonists. To measure whether BM-SMC exhibited functional properties of mature V-SMC, an isolated tissue bath was used to measure the isometric tension generated by segments of cylindrical BM-TEV that were cultured for two weeks.

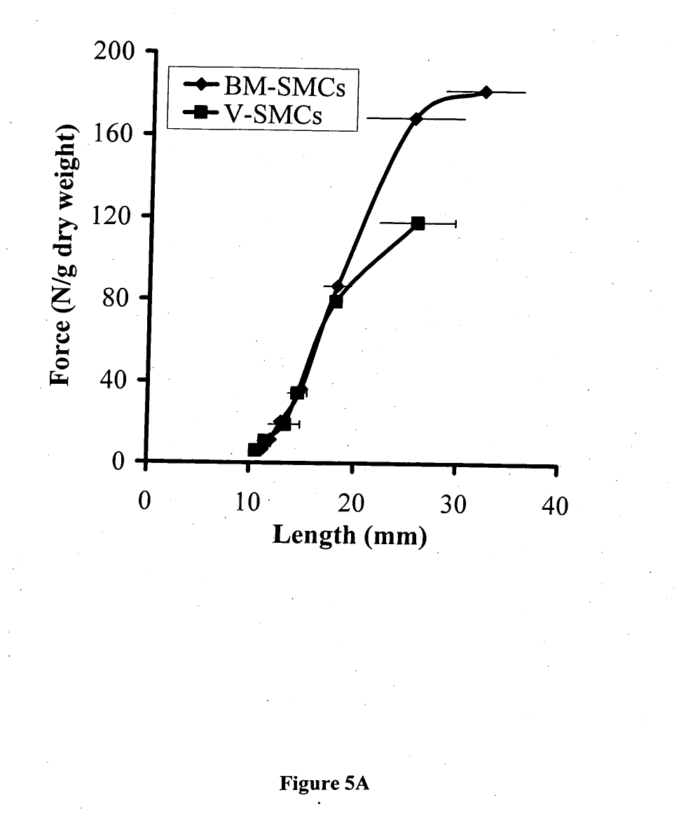

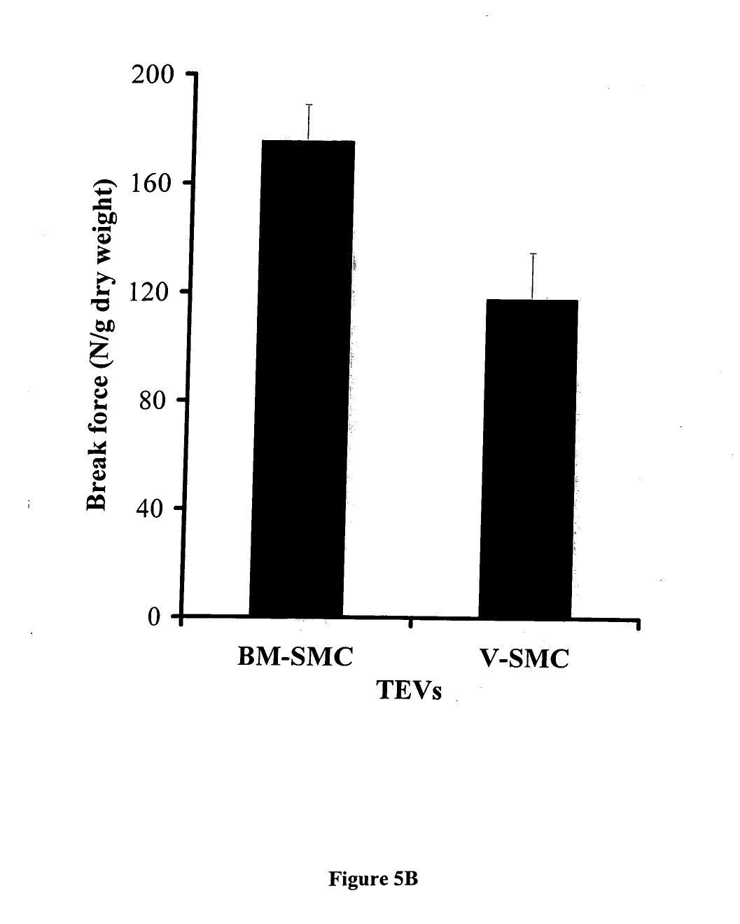

BM-TEV exhibited enhanced mechanical properties as compared to TEV from V-SMC. Specifically, BM-TEV showed significantly higher break force and toughness but similar elastic modulus as TEV from V-SMC (FIGS. 5A-D). In addition, both tissues showed active pathways of receptor and non-receptor mediated vascular reactivity. Specifically, BM-TEV exhibited vasoconstriction in response to KCl (118 mM) or NE (10⁻⁶ M) to the same extent as TEV from V-SMC (FIG. 5E). In contrast, TEV generated from unsorted BM-MNC showed no reactivity in response to KCl or NE, suggesting that only a small fraction of BM-MNC with an active SMα-actin promoter exhibited functional properties of mature SMC.

### Example 14

### BM-TEV Exhibited High Potential for Tissue Remodeling In Vivo

Next it was hypothesized that BM-TEV may show improved ability for remodeling after implantation in vivo. To address this hypothesis, bi-layered BM-TEV were prepared by sequential layering of two fibrin hydrogels in a concentric cylindrical arrangement, as described recently. First, a cell-free fibrin layer containing high FBG concentration (30 mg/ml) was formed around a 4.0 mm cylindrical mandrel. A second fibrin layer containing SMC (1×10⁶ cells/mL) in low FBG (2.5 mg/ml) was polymerized around the first layer. These tissues displayed similar vascular reactivity as single-layered TEV, but were significantly stronger, exhibiting a burst pressure 180 mmHg.

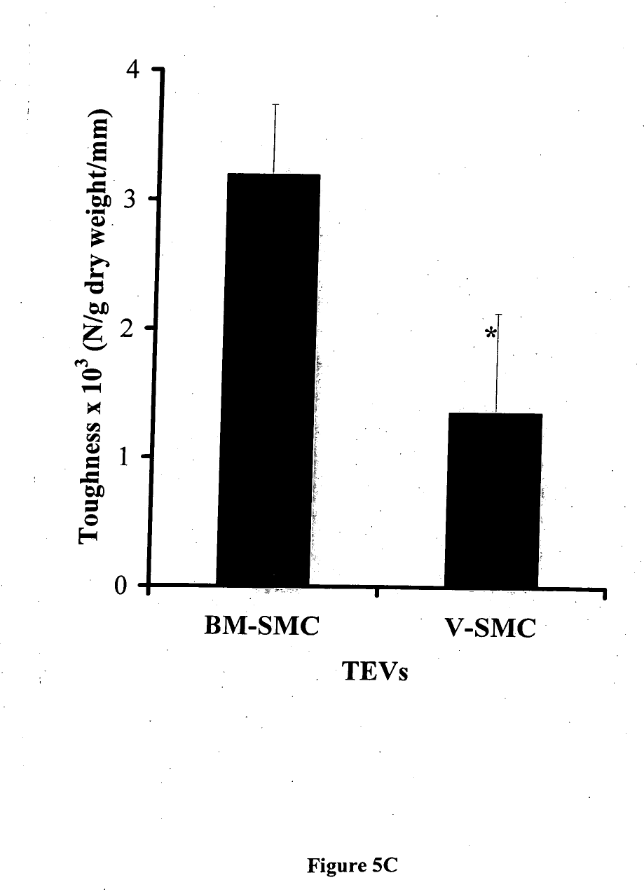

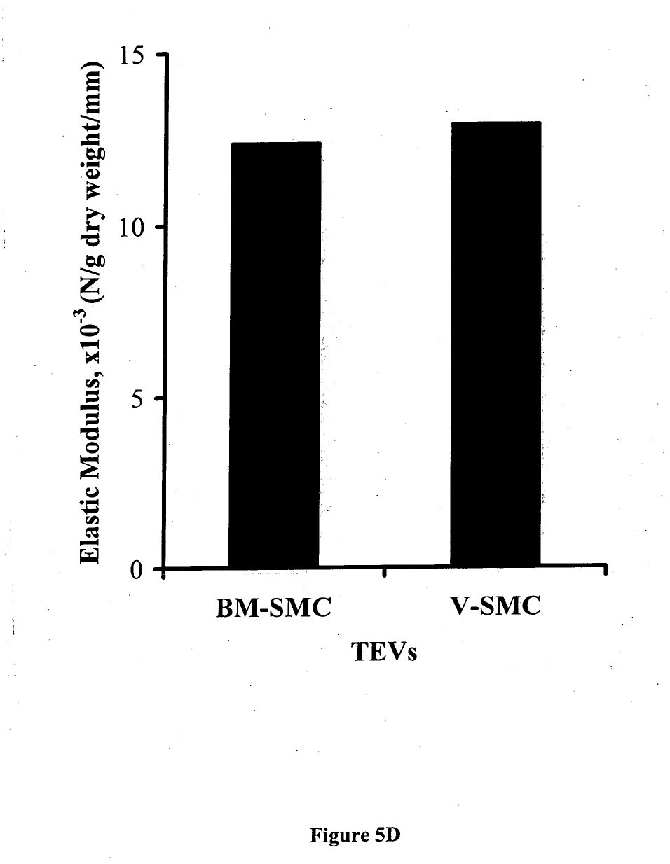

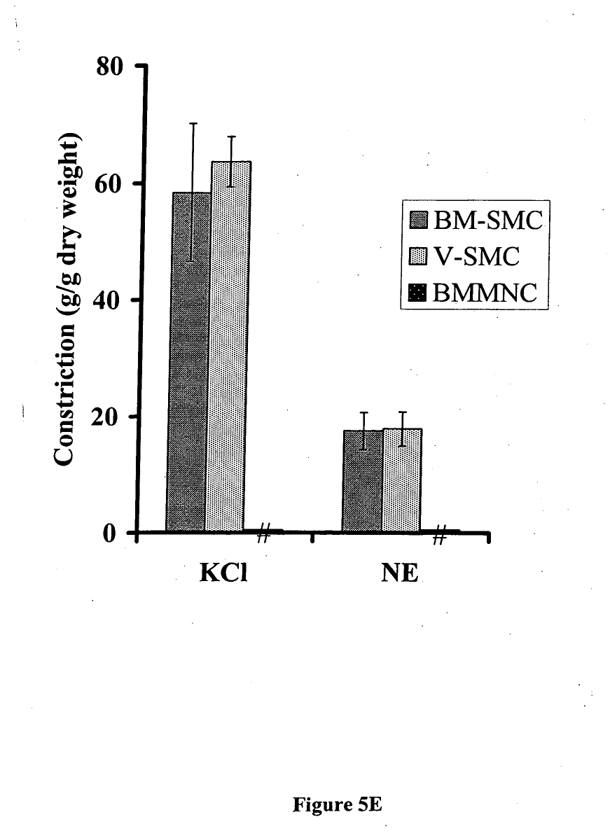

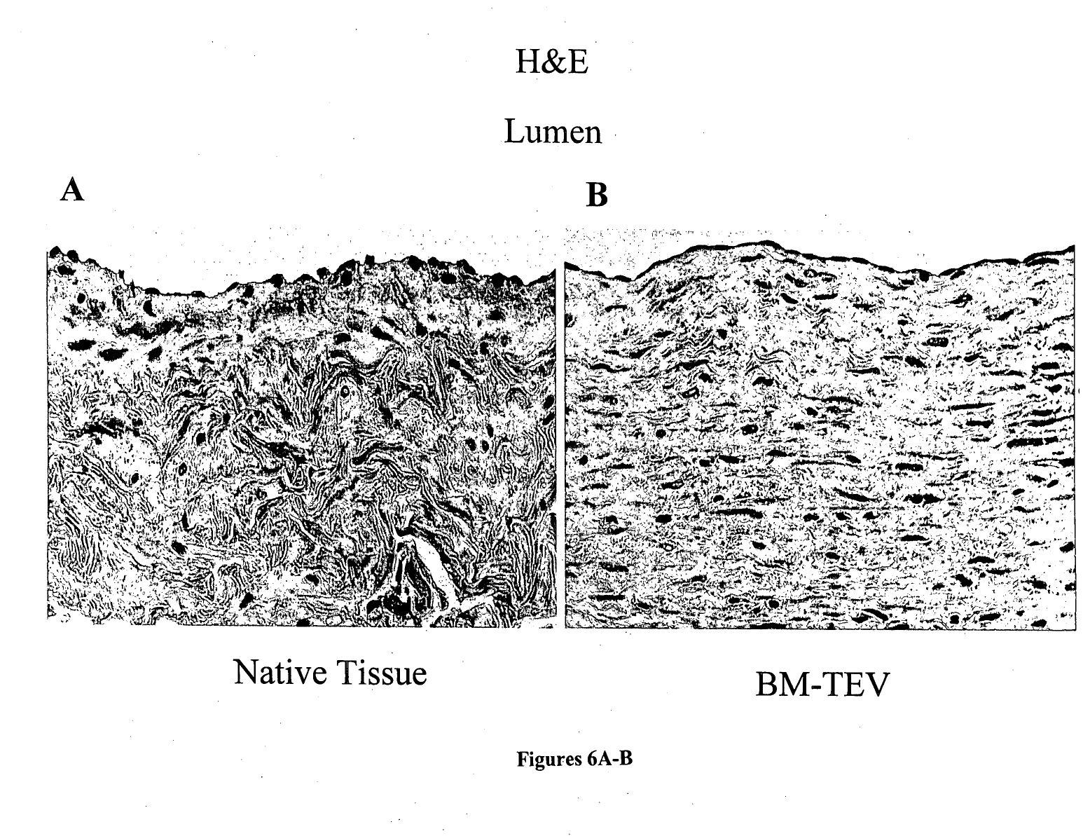

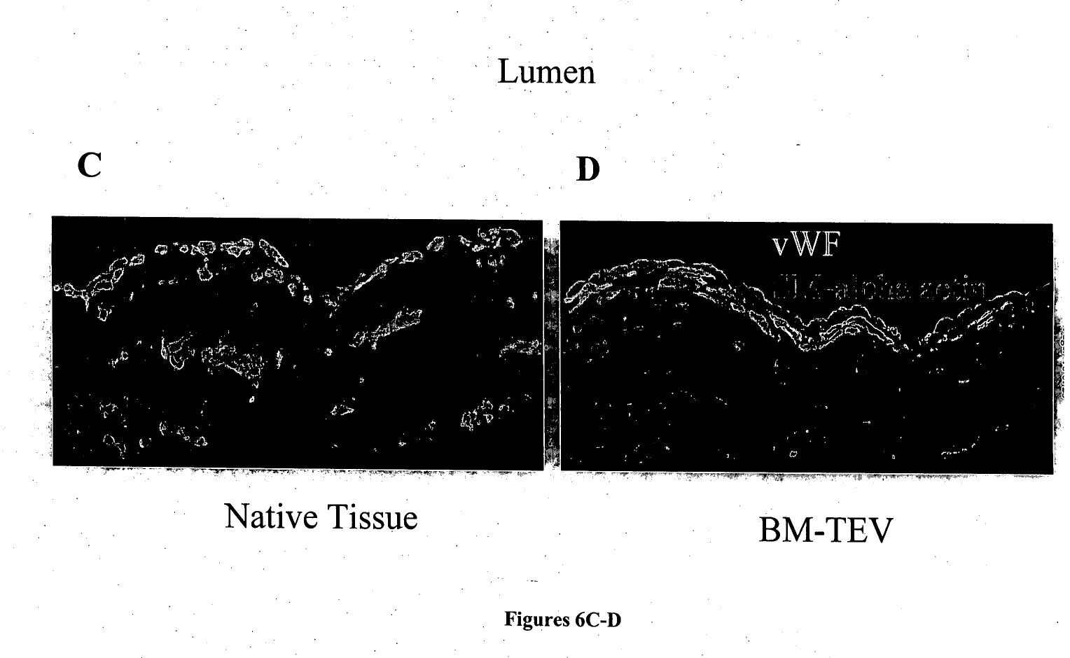

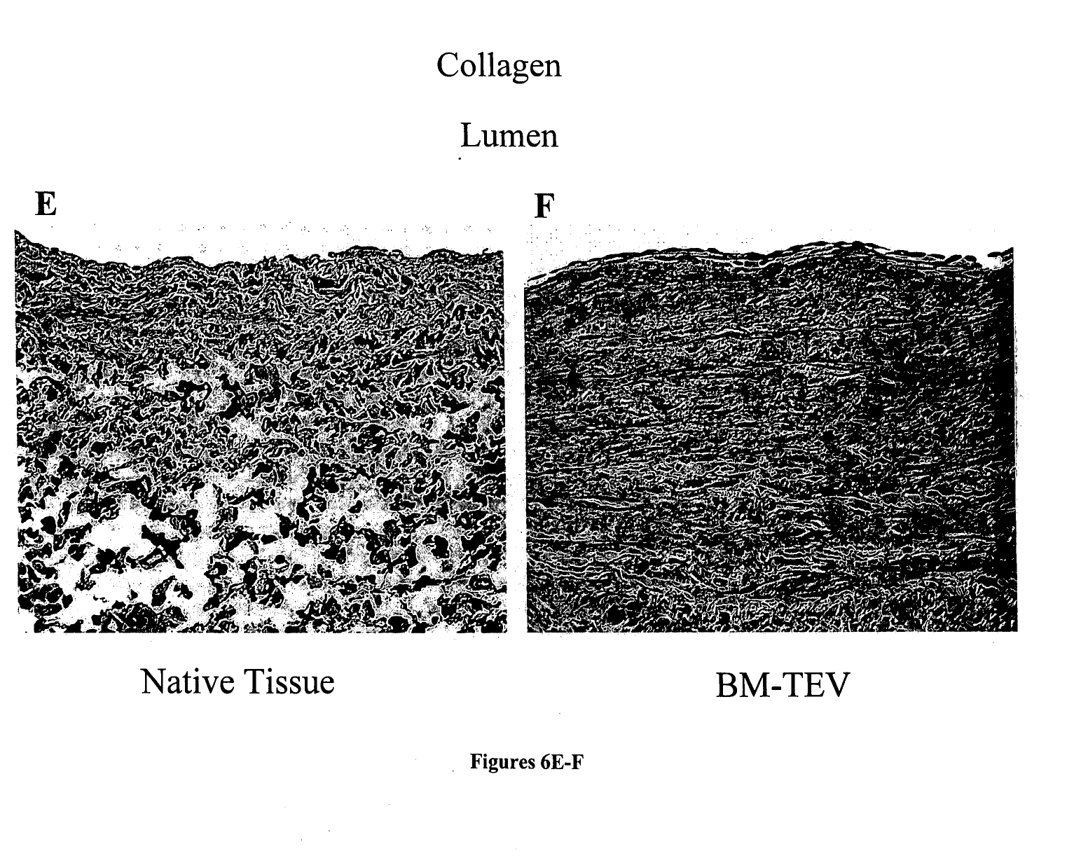

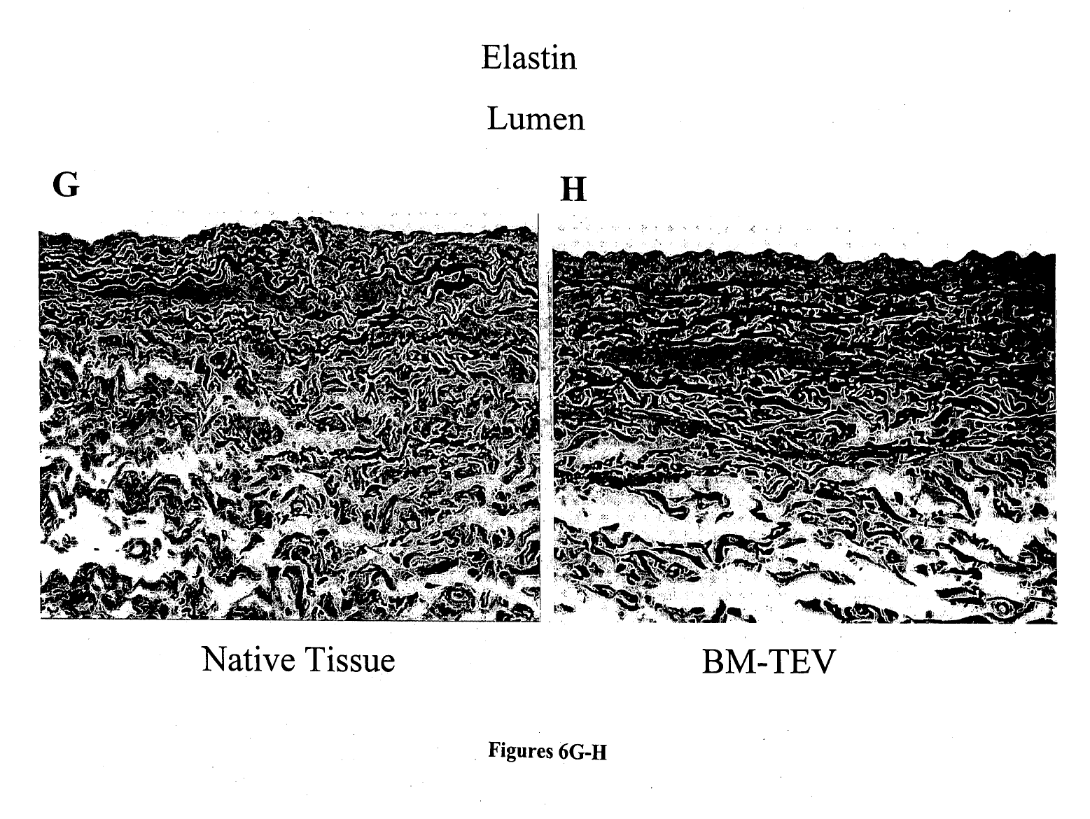

After two weeks in culture the tissues were removed from the mandrels, BM-EC were seeded in the lumen, and cultured for 10 more days before interpositional implantation into the jugular vein of 8 week old lambs. Patency was determined by angiography, which showed one completely open and one partially occluded vessel after 5 or 8 weeks, respectively. After angiography, BM-TEV were removed from the animals and processed for histology (n=2). Hematoxylin-eosin stain showed that, similar to native jugular vein (FIG. 6A), explanted BM-TEV (FIG. 6B) contained multiple layers of SMC which were overlaid by a continuous monolayer of endothelial cells. Immunostaining showed that the intimal and medial layers were stained for vWF and smooth muscle α-actin, respectively (FIGS. 6C-D). BM-SMC in close proximity to the lumen assumed circumferential orientation and produced a high amount of collagen matrix throughout the tissue (FIGS. 6E-F). Most important, implanted tissues contained significant amounts of elastin that displayed fibrillar organization just as in the native tissue (FIGS. 6G-H). In contrast, TEV from mature V-SMC exhibited very low amount of elastin synthesis and no fiber organization, even after 15 weeks in vivo (Swartz et al., “Engineering of Fibrin-Based Functional and Implantable Small-Diameter Blood Vessels,” Am. J. Physiol. Heart Circ. Physiol. 288(3):H1451-1460 (2005), which is hereby incorporated by reference in its entirety), indicating that BM-SMC may have a higher elastogenic potential than mature V-SMC.

Lack of availability of autologous vascular grafts and the pain and discomfort associated with the donor site necessitate the development of tissue engineered blood vessels for tissue regeneration. A novel method for isolation of BM-SMC from bone marrow progenitors using a tissue specific promoter, SMαA, driving expression of EGFP has been shown. BM-SMC showed high proliferative potential and displayed morphological and phenotypic properties of V-SMC as shown by expression of smooth muscle markers such as α-actin and calponin. Notably, BM-SMC displayed contractile properties suggesting that these cells had developed a functionally mature SMC phenotype. Most important, TEV engineered from BM-derived SMC and EC were implanted into the jugular vein of an ovine animal model and demonstrated remarkable ability for matrix remodeling as evidence by production of collagen and elastin fibers.

Contractility is the defining property of mature SMC and one of the most important properties of blood vessels. When BM-SMC were embedded in fibrin hydrogels they compacted the gels to approximately 5% of their original volume within 3 days in culture. Most important, BM-TEV displayed vascular reactivity in response to vasoconstrictors such as KCl and NE. Since KCl causes contraction by opening the L-type, slow calcium potential-dependent channels while NE acts through α1 and α2 receptors, these results demonstrate that BM-SMC had developed both receptor and non-receptor mediated pathways of vascular reactivity. In contrast, TEV prepared from unsorted BM-MNCs displayed no contractility, indicating that the complex and heterogeneous bone marrow microenvironment contains a small fraction of functional SMC, which retain their biochemical and contractile properties after purification and expansion in vitro.

Functional BM-SMC and BM-EC were used to engineer small diameter blood vessels that were implanted into the jugular veins of lambs. Histology showed that the morphology and cellular organization of the explanted BM-TEV was very similar to that of native tissues. The SMC close to the lumen appeared to be circumferentially aligned and displayed highly organized fibers of α-actin. The endothelial monolayer in the lumen appeared to be confluent and expressed high quantities of vWF. BM-SMC remodeled fibrin and expressed high amounts of collagen throughout the medial layer. Most notably, BM-SMC expressed significant amount of highly organized elastin fibers, very similar to the native tissue. In contrast, TEV from V-SMC expressed significantly smaller quantities of elastin even at 15 weeks post-implantation (Swartz et al., “Engineering of Fibrin-Based Functional and Implantable Small-Diameter Blood Vessels,” Am. J. Physiol. Heart Circ. Physiol. 288(3):H1451-1460 (2005), which is hereby incorporated by reference in its entirety), suggesting that BM-SMC may be better endowed to remodel the implanted tissues and contribute to their long-term function and mechanical stability.

### Example 15

### Engineering of Functional Blood Vessels from Hair Follicle Stem Cells

Safe and less invasive harvest of functional smooth muscle cells with tremendous proliferating potential offer practical optional treatment for diseases involving smooth muscle cell pathology, such as cardiovascular disease.

Hair follicle contains stem cells and is easily accessible. Harvest of autologous stem cells from hair follicle is less invasive and requires less manipulation than the harvest of stem cells from other cells sources, such as cord blood, peripheral blood, bone marrow, adipose tissue, testis, brain, and eye.

Hair follicle-derived smooth muscle precursor cells from anagen hair exhibit significant proliferating potential, independent of age and sex, thus providing nearly unlimited cell sources for cell-based tissue engineering and regenerative medicine.

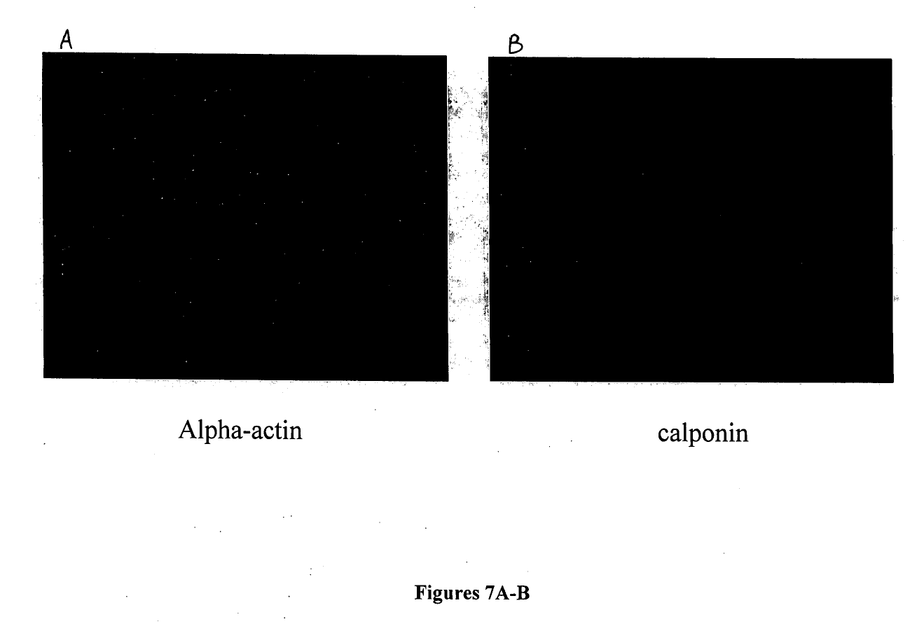

Smooth muscle precursor cells were isolated from hair follicle (“HF-SMC”). HF-SMC were shown to express α-actin and calponin, the specific markers of vascular smooth muscle, as by immunocytochemistry (FIGS. 7A-B).

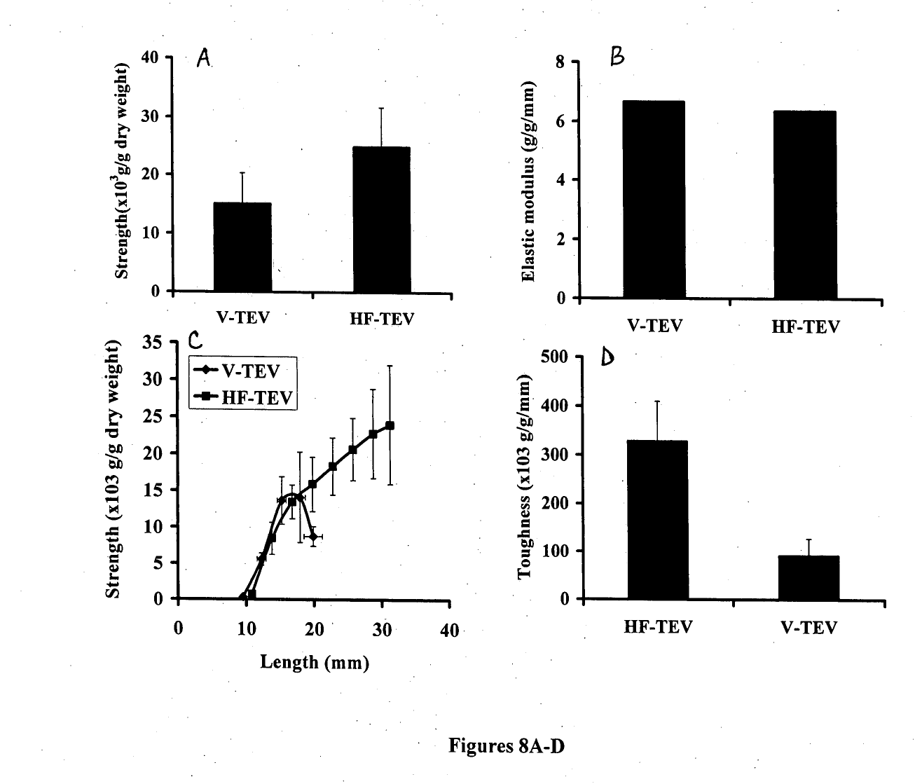

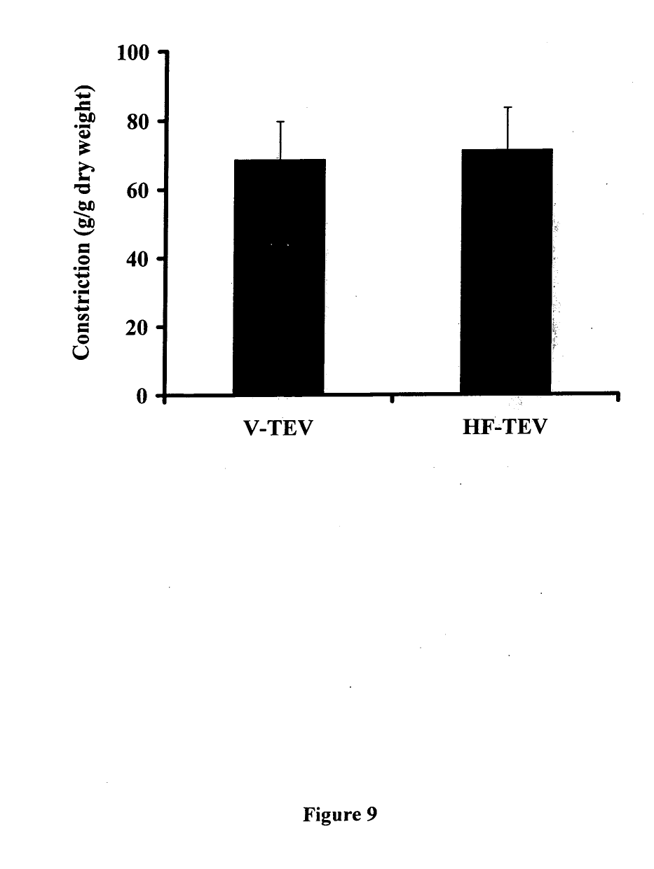

Blood vessels were tissue-engineered using HF-SMC as cell sources and fibrin hydro gel as a scaffold. HF-SMC derived TEVs (“HF-TEV”) demonstrated significant mechanical properties and vasoreactivity to KCl, comparable to TEVs from vascular smooth muscle cells (“V-TEV”) (FIGS. 8A-D). HF-TEV also demonstrated significant contractility (the quintessential property of smooth muscle cells), comparable to V-TEVs (FIG. 9).

Although the invention has been described in detail for the purposes of illustration, it is understood that such detail is solely for that purpose, and variations can be made therein by those skilled in the art without departing from the spirit and scope of the invention which is defined by the following claims.

What is claimed:

1. A method of isolating smooth muscle cells or progenitors thereof from a mixed population of cells, said method comprising:
selecting an enhancer/promoter which functions in said smooth muscle cells or progenitors thereof;
introducing a nucleic acid molecule encoding a marker protein under control of said enhancer/promoter into the mixed population of cells;
allowing the smooth muscle cells or progenitors thereof to express the marker protein; and
separating the smooth muscle cells or progenitors thereof from the mixed population of cells based on expression of the marker protein.

2. The method according to claim 1, wherein said mixed population of cells are bone marrow-derived cells.

3. The method according to claim 1, wherein said mixed population of cells are hair follicle-derived cells.

4. The method according to claim 1, wherein said enhancer/promoter is a smooth muscle α-actin promoter.

5. The method according to claim 1, wherein said introducing comprises transfection of said mixed population of cells.

6. The method according to claim 1, wherein said marker protein is a fluorescent protein.

7. The method according to claim 6, wherein said separating F comprises fluorescence activated cell sorting.

8. The method according to claim 1, wherein the smooth muscle cells or progenitors thereof are human cells.

9. The method according to claim 1, wherein the smooth muscle cells or progenitors thereof are of adult origin.

10. A preparation of isolated smooth muscle cells or progenitors thereof, wherein the smooth muscle cells or progenitors thereof comprise at least 90% of said preparation.

11. The preparation according to claim 10, wherein the smooth muscle cells are bone marrow-derived smooth muscle cells.

12. The preparation according to claim 10, wherein the smooth muscle cells are hair follicle-derived smooth muscle cells.

13. The preparation according to claim 10 comprising smooth muscle cells.

14. The preparation according to claim 10 comprising smooth muscle progenitor cells.

15. The preparation according to claim 10, wherein the smooth muscle cells or progenitors thereof comprise at least 95% of said preparation.

16. The preparation according to claim 10, wherein the smooth muscle cells or progenitors thereof are human cells.

17. The preparation according to claim 10, wherein the cells are of adult origin.

18. A method of producing a tissue-engineered vascular vessel comprising:
providing a vessel-forming fibrin mixture comprising fibrinogen, thrombin, and the preparation of isolated smooth muscle cells or progenitors thereof according to claim 10;
molding the vessel-forming fibrin mixture into a fibrin gel having a tubular shape; and
incubating the fibrin gel having a tubular shape in a medium suitable for growth of the cells under conditions effective to produce a tissue-engineered vascular vessel.

19. The method according to claim 18, wherein said molding is carried out in a tube with an inner mandrel.

20. The method according to claim 19, wherein the vessel has an interior surface, said method further comprising:
seeding endothelial cells on the interior surface of the vessel.

21. The method according to claim 18 further comprising:
subjecting the fibrin gel having a tubular shape to a pulse after said molding.

22. The method according to claim 18 further comprising:
combining the fibrin gel with a porous scaffold prior to said incubating.

23. The method according to claim 22, wherein the porous scaffold is selected from the group consisting of decellularized elastin, poly lactic-glycolic acid, and mixtures thereof.

24. A tissue-engineered vascular vessel comprising:
a gelled fibrin mixture comprising fibrinogen, thrombin, and the preparation of isolated smooth muscle cells or progenitors thereof according to claim 10, wherein the gelled fibrin mixture has a tubular shape.

25. The tissue-engineered vascular vessel according to claim 24, wherein the gelled fibrin mixture contains a porous scaffold.

26. The tissue-engineered vascular vessel according to claim 25, wherein the porous scaffold is selected from the group consisting of decellularized elastin, poly lactic-glycolic acid, and mixtures thereof.

27. A method of producing a tissue-engineered vascular vessel for a particular patient comprising:
providing a vessel-forming fibrin mixture comprising fibrinogen, thrombin, and the preparation of isolated smooth muscle cells or progenitors thereof according to claim 10, at least one of which is autologous to the patient;
molding the vessel-forming fibrin mixture into a fibrin gel having a tubular shape;
incubating the fibrin gel having a tubular shape in a medium suitable for growth of the cells under conditions effective to produce a tissue-engineered vascular vessel for a particular patient; and
implanting the tissue-engineered vascular vessel into the particular patient.
